SEQUENCE LISTING (16 sequences)

SEQ ID NO: 1 - DNA, 42 bp, Artificial sequence
  Synthetic oligonucleotide used for PCR amplification of individual sugar biosynthetic gene
agagaagcttactagtgcccccccgagaggcagcggcccatg

SEQ ID NO: 2 - DNA, 42 bp, Artificial sequence
  Synthetic oligonucleotide used for PCR amplification of individual sugar biosynthetic gene
agtctagagctagctcatgctgctcctcgccgggtcggtggg

SEQ ID NO: 3 - DNA, 43 bp, Artificial sequence
  Synthetic oligonucleotide used for PCR amplification of individual sugar biosynthetic gene
agagaagcttgctagccggaccacgcgaaggacctttcacatg

SEQ ID NO: 4 - DNA, 45 bp, Artificial sequence
  Synthetic oligonucleotide used for PCR amplification of individual sugar biosynthetic gene
agtctagattaattaagctagttgtcgttccagaacggctcccgg

SEQ ID NO: 5 - DNA, 37 bp, Artificial sequence
  Synthetic oligonucleotide used for PCR amplification of individual sugar biosynthetic gene
acgtaagcttcctagggcgccgtcctggatcacaatg

SEQ ID NO: 6 - DNA, 36 bp, Artificial sequence
  Synthetic oligonucleotide used for PCR amplification of individual sugar biosynthetic gene
acgtctagagttaaccttcggggtgctcagctcagg

SEQ ID NO: 7 - DNA, 36 bp, Artificial sequence
  Synthetic oligonucleotide used for PCR amplification of individual sugar biosynthetic gene
acgtaagcttgttaacccgaagggaaccccatgccc

SEQ ID NO: 8 - DNA, 36 bp, Artificial sequence
  Synthetic oligonucleotide used for PCR amplification of individual sugar biosynthetic gene
acgtctagaactagtgcatcagcaccagcgcacccg

SEQ ID NO: 9 - DNA, 47 bp, Artificial sequence
  Synthetic oligonucleotide used for PCR amplification of individual sugar biosynthetic gene
agagaagcttactagtgggtcacgcggagacgggtgaggcggacatg

SEQ ID NO: 10 - DNA, 40 bp, Artificial sequence
  Synthetic oligonucleotide used for PCR amplification of individual sugar biosynthetic gene
agtctagagctagcctaggccggggcgccgtgcgcggtcc

SEQ ID NO: 11 - DNA, 45 bp, Artificial sequence
  Synthetic oligonucleotide used for PCR amplification of individual sugar biosynthetic gene
agagaagcttactagtgcgtgctggtcaccggaggagaaggcatg

SEQ ID NO: 12 - DNA, 42 bp, Artificial sequence
  Synthetic oligonucleotide used for PCR amplification of individual sugar biosynthetic gene
agtctagagctagctcacgtccccgtcaggttcctgaggcgc

SEQ ID NO: 13 - DNA, 43 bp, Artificial sequence
  Synthetic oligonucleotide used for PCR amplification of individual sugar biosynthetic gene
agagaagcttactagtcgctccgcccaccgggcgcgacgcatg

SEQ ID NO: 14 - DNA, 42 bp, Artificial sequence
  Synthetic oligonucleotide used for PCR amplification of individual sugar biosynthetic gene
agtctagagctagctcacgagtggggtgcccgcgacggccgg

SEQ ID NO: 15 - DNA, 48 bp, Artificial sequence
  Synthetic oligonucleotide used for PCR amplification of individual sugar biosynthetic gene
agagaagcttactagtcagtgacgggttgccgcacatcgcgctgaatg

SEQ ID NO: 16 - DNA, 45 bp, Artificial sequence
  Synthetic oligonucleotide used for PCR amplification of individual sugar biosynthetic gene
agtctagagctagcctagtgctcctcggtgggggtcagggcggcc

(19) United States
(12) Patent Application Publication
(10) Pub. No.: US 2007/0059689 A1
(43) Pub. Date: Mar. 15, 2007

(54) Hybrid glycosylated products and their production and use

(76) Inventors: Ignacio Aguirrezabalaga, Oveido (ES); Nerea Allende, Dundee, SCT (US); Alfredo F. Brana, Oviedo (ES); Carmen Mendez, Oviedo (ES); Leticia Rodriquez, Martinsried (DE); Jose A. Salas, Oviedo (ES)

Correspondence Address: KLAUBER & JACKSON, 411 HACKENSACK AVENUE, HACKENSACK, NJ, 07601, US

(21) Appl. No.: 10/496,751

(22) PCT Filed: Nov. 29, 2002
(86) PCT No.: PCT/GB02/05426

§ 371 Date: Feb. 28, 2005

(30) Foreign Application Priority Data
Nov. 29, 2001 (GB) 0128680.6

(51) Int. Cl. C12Q 1/68; C07H 21/04; C12P 21/06; C07K 14/195

(52) U.S. Cl. 435/6; 435/69.1; 435/252.3; 435/320.1; 530/395; 536/23.2

(57) ABSTRACT

Hybrid glycosylated products such as polyketides and peptides are produced by transforming a host cell with (a) a gene cassette for synthesising an activated sugar and (b) nucleic acid encoding a glycosyltransferase (GT). The cell also produces or is supplied with an aglycone template. At least some of the components (sugar, aglycone, GT, sugar synthesis genes, cells) are mutually heterologous.

### FIELD OF THE INVENTION

The present invention relates to hybrid glycosylated products, and in particular, to natural products such as polyketides and glycopeptides, and to processes for their preparation. The invention is particularly concerned with cells containing cloned sets of biosynthetic genes for specific activated deoxysugars in a cassette format, housed for example on a plasmid. The cassette format allows convenient addition, removal or replacement of the genes in the sugar cassette so as to produce a combinatorial library of activated deoxysugars. In such cells a cloned microbial glycosyltransferase can be conveniently tested for its ability to generate specific glycosylated derivatives when supplied with polyketide, polypeptide, or polyketide-polypeptides as substrates.

### BACKGROUND TO THE INVENTION

Glycosylation is important for the bioactivity of many natural products, including antibacterial compounds such as the polyketide erythromycin A and the glycopeptide vancomycin, and antitumour compounds such as the aromatic polyketide daunorubicin and the glycopeptide-polyketide bleomycin. Polyketides are a large and structurally diverse class of natural products that includes many compounds possessing antibiotic or other pharmacological properties, such as erythromycin, tetracyclines, rapamycin, avermectin, monensin, epothilones and FK506. In particular, Streptomyces and related genera are prodigious producers of polyketides. Polyketides are synthesised by the repeated stepwise condensation of acylthioesters in a manner analogous to that of fatty acid biosynthesis (Rawlings, 1999 and 2001a&b). The greater structural diversity found among natural polyketides arises from the selection of acyl-CoA starter units, and (generally) malonyl-CoA, methylmalonyl-CoA and ethylmalonyl-CoA as extender units; and from the differing degree of processing of the β-keto group observed after each condensation. Examples of processing steps include reduction to β-hydroxyacyl-, reduction followed by dehydration to 2-enoyl-, and complete reduction to the saturated acylthioester. The stereochemical outcome of these processing steps is also specified for each cycle of chain extension. The polyketide chains are in many cases cyclised in specific ways and subject to further enzyme-catalysed modifications to produce the final polyketide. Naturally-occurring peptides produced by non-ribosomal peptide synthetases are likewise synthesised by repeated stepwise assembly, in this case of activated amino acids, and the chains produced are similarly subject to further modifications to produce the fully bioactive molecules (von Döhren et al., 1997). Mixed polyketide-peptide compounds, hereinafter defined as incorporating both ketide and amino acid units, are also known and their bioactivity is also influenced by their pattern of glycosylation and other modification (Du et al., 2001). The compounds produced by these related pathways are particularly valuable because they include large numbers of compounds of known utility, for example as anthelminthics, insecticides, immunosuppressants, antifungal or antibacterial agents.

Although large numbers of therapeutically important polyketides have been identified, there remains a need to obtain novel polyketides that have enhanced properties or that possess completely novel bioactivity. The inexorable rise in the incidence of pathogenic organisms with resistance to antibiotics such as 14-membered macrolides or glycopeptides represents a significant threat to human and animal health. Current methods of obtaining novel polyketide metabolites include large-scale screening of naturally-occurring strains of Streptomyces and other organisms, either for direct production of useful molecules, or for the presence of enzymatic activities that can bioconvert, to specific derivatives, a known polyketide added to the growth medium. These procedures are time-consuming and costly, and biotransformation using whole cells may in addition be limited by side-reactions or by a low concentration or activity of the intracellular enzyme responsible for the bioconversion. Given the complexity of bioactive polyketides, they are not readily amenable to total chemical synthesis in large scale. Chemical modification of existing polyketides has been widely used, but many desirable alterations are not readily achievable by these means.

Methods have been developed for the biosynthesis of altered polyketides and non-ribosomally-synthesised polypeptides by the engineering of the corresponding genes encoding the polyketide synthases and polypeptide synthetases respectively. The biosynthesis of polyketides is initiated by a group of chain-forming enzymes known as polyketide synthases. Three classes of polyketide synthase (PKS) have been described in actinomycetes.

The first class, named Type I PKSs (Rawlings, 2001a&b) and represented by the PKSs for the macrolides erythromycin, oleandomycin, avermectin and rapanycin, consists of a different set or “extension module” of enzymes for each cycle of polyketide chain extension (Cortés et al., 1990). The term “extension module” as used herein refers to the set of contiguous domains, from a β-ketoacyl-ACP synthase (“KS”) domain to the next acyl carrier protein (“ACP”) domain, which accomplishes one cycle of polyketide chain extension.

The modular arrangement of type-I PKSs was first confirmed by mutation of the erythromycin PKS (also known as 6-deoxyerythronolide B synthase, DEBS) through an in-frame deletion of the DNA encoding part of the ketoreductase domain in module 5 (Donadio et al., 1991). This led to the production of the erythromycin analogues 5,6-dideoxy-3-β-mycarosyl-5-oxoerythronolide B and 5,6-dideoxy-5-oxoerythronolide B due to the inability of the mutated KR domain of module 5 to reduce the β-keto group introduced at this stage of the processive biosynthesis. Likewise, alteration of active site residues in the enoylreductase domain of module 4 in DEBS, by genetic engineering of the corresponding PKS-encoding DNA and its introduction into Saccharopolyspora erythraea, led to the production of 6,7-anhydroerythromycin C (Donadio et al., 1993). WO 93/13663 describes additional types of genetic manipulation of the DEBS genes that are capable of producing altered polyketides. However, many such attempts are reported to have been unproductive (Hutchinson & Fujii, 1995).

WO 98/01546 describes the engineering of hybrid Type I PKS genes which utilise portions of PKS genes derived from more than one natural PKS, particularly derived from different organisms, and the use of such recombinant genes for the production of altered polyketide metabolites.

The second class of PKS, named Type II PKSs, is represented by the synthases for many aromatic compounds produced by bacteria. Type II PKSs contain only a single set of enzymatic activities for chain extension and these are re-used in successive cycles (Bibb et al., 1989; Sherman et al., 1989; Fernandez-Moreno et al., 1992). The extender units for the Type II PKSs are usually malonyl-CoA units, and the presence of specific cyclases dictates the preferred pathway for cyclisation of the completed chain into an aromatic product (Hutchinson & Fujii, 1995). Hybrid polyketides have been obtained by the introduction of cloned Type II PKS gene-containing DNA into another strain containing a different Type II PKS gene cluster, for example, by introduction of DNA derived from the gene cluster for actinorhodin, a blue-pigmented polyketide from Streptomyces coelicolor, into an anthraquinone polyketide-producing strain of Streptomyces galileus (Bartel et al., 1990).

The minimal number of domains required for polyketide chain extension on a Type II PKS when expressed in a Streptomyces coelicolor host cell (the “minimal PKS”) has been defined (for example in WO 95/08548) as containing the following three polypeptides which are products of the act I genes: first a KS; secondly a polypeptide termed the CLF with end-to-end amino acid sequence similarity to the KS but in which the essential active site residue of the KS, namely a cysteine residue, is substituted either by a glutamine residue, or in the case of the PKS for a spore pigment such as the whiE gene product (Chater & Davis, 1990) by a glutamic acid residue; and finally an ACP. The CLF has been stated (for example in WO 95/08548) to be a factor that determines the chain length of the polyketide chain that is produced by the minimal PKS. However, it has been found (Shen et al., 1995) that when the CLF for the octaketide actinorhodin is used to replace the CLF for the decaketide tetracenomycin in host cells of Streptomyces glaucescens, the polyketide product is not found to be altered from a decaketide to an octaketide. An alternative nomenclature has been proposed in which KS is designated KS_{α} and CLF is designated KS_{β} to reflect this lack of confidence in the correct assignment of the function of CLF (Meurer et al., 1997). WO 00/00618 has recently shown that CLF and its counterpart in Type I PKS multienzymes, the so-called KSQ domain, are involved in initiation of polyketide chain synthesis. WO 95/08548 for example describes the replacement of actinorhodin PKS genes by heterologous DNA from other Type II PKS gene clusters, to obtain hybrid polyketides.

The third class of PKS, named Type III PKSs, is represented by the synthases for certain other aromatic compounds in bacteria, such as flaveolin from Streptomyces griseus (Funa et al., 1999). Type III PKSs contain only a single active site where chain extension occurs. The “extender” units for the Type III PKSs are usually acetate units, and the shape and volume of the active site apparently dictate the preferred pathway for cyclisation of the completed chain into an aromatic product (Jez et al., 2000). The product of a Type III PKS has been successfully altered by specific mutagenesis of portions of the enzyme structure, based on a knowledge of the X-ray crystal structure of typical chalcone synthases and stilbene synthases (Ferrer et al., 1999). The aromatic products of Type III PKSs are often further processed by oxidation, and deoxsugar attachement may also occur (Cortes et al., 2002).

This ability to engineer PKS genes of all Type I, Type II and Type III origin raises the possibility of the combinatorial biosynthesis of polyketides to produce diverse libraries of novel natural products which may be screened for desirable bioactivities. However, the aglycones produced by recombinant PKS genes may or may not be partially processed by glycosyltransferases and/or other modifying enzymes into analogues of the mature polyketides. There is therefore an additional need to provide processes for efficient conversion of such novel aglycones into specific glycosylated products. Further, the invention of efficient processes for glycosylation would provide a new means to increase very significantly the diversity of combinatorial polyketide libraries, by utilisation of recombinant cells containing alternative cloned glycosyltransferases and alternative complements of activated sugars (Salas and Méndez, 2001; Rohr et al., 2002).

The well-known influence of glycosylation on biological activity has encouraged intensive research into the genes and enzymes governing the synthesis and attachment of specific sugar units to polyketide and polypeptide metabolites (for a review see Trefzer et al., 1999). Surveys of such metabolites have revealed a high diversity in the type of glycosyl substitution that is found, including a very large number of different deoxyhexoses and deoxyaminohexoses (see for a review Liu & Thorson, 1994). The sequencing of biosynthetic gene clusters for numerous glycosylated polyketides, polypeptides and mixed polyketide/peptide systems has revealed the presence of such sugar biosynthetic genes. In addition, genes encoding the glycosyltransferases that transfer the glycosyl group from an activated form of the sugar, eg dTDP- or dUDP- forms, to the aglycone acceptor have also been identified. For example, the eryB genes and the eryC genes of the erythromycin biosynthetic gene cluster in Saccharopolyspora erythraea have been identified as involved in the biosynthesis and attachment to the aglycone precursor of erythromycin A of L-mycarose and D-desosamine, respectively (Dhillon et al., 1989; Haydock et al., 1991; Salah-Bey et al., 1998; Gaisser et al., 1998; Gaisser et al., 1997; Summers et al., 1997). Both WO 97/23630 and WO 99/05283 describe the preparation of an altered erythromycin by deletion of a specific sugar biosynthetic gene, so that an altered sugar becomes attached to the aglycone. Thus WO 99/05283 describes low but detectable levels of erythromycins in which, for example, desosamine is replaced by mycaminose (eryCIV knockout), or desmethylmycarosyl erythromycins (eryBIII knockout) are produced. Meanwhile methymycin analogues have been produced in which desosamine has been replaced by D-quinuvose (Borisova et al., 1999) or which display altered patterns of amino group substitution through the incorporation of the calH gene of the calicheamycin gene cluster from Micromonospora echinospora into the methymycin producing strain (Zhao et al., 1999). Similarly, hybrid glycopeptides have been produced by using cloned glycosyltransferases from the vancomycin-producer Amycolatopsis orientalis to add D-xylose or D-glucose to aglycones of closely-related glycopeptides according to U.S. Pat No. 5,871,983 (see also Solenberg et al., 1997). Hybrid aromatic polyketides have also been produced by interspecies complementation, by utilising a homologue of a biosynthetic gene that performs an analogous chemical reaction, but with a different stereospecificity. Thus, instead of the natural daunosamine, 4′epi-daunosamine is produced in recombinant Streptomyces peucetius and attached by the daunosamine glycosyltransferase to the natural aglycone, to yield the antitumour derivative epirubicin in place of doxorubicin (Madduri et al., 1998).

In all these cases, the broad specificity of the glycosyltransferase allowed the substitution of an alternative activated sugar, but the aglycone and glycosyltransferase were not heterologous to each other. It has been found that when the oleandrose glycosyltransferase OleG2, from the oleandomycin biosynthetic gene cluster of Streptomyces antibioticus, is cloned into the erythromycin-producing strain S. erythraea, in addition to other products, a novel erythromycin in which the L-cladinose moiety at C-3 is replaced by L-rhamnose was obtained (Doumith et al., 1999). It was assumed that the activated L-rhamnose is produced by the host cells, and is recruited by OleG2 in competition with the activated L-mycarose known to be present.

The sugar D-desosamine is found attached to many macrolides (Trefzer et al., 1999) and it has been found that biosynthetic genes for the biosynthesis of the activated form of this sugar are accordingly found in each of the gene clusters for such macrolides. Similarly the sugar L-oleandrose is found for example in oleandomycin produced by S. antibioticus, while two such oleandrosyl moieties are present in avermectins produced by Streptomyces avermitilis. Comparison of gene sequences and specific gene disruptions has led to the recognition that in S. avermitilis eight genes from the avermectin cluster (avrBCDEFGHI) are involved in the biosynthesis and attachment of L-oleandrose (for a review see Wohlert et al., 2001). The AvrD and AvrC enzymes control production of dTDP-4-keto-6-deoxyglucose; C-2 deoxygenation occurs next catalyzed by AvrG and AvrI; it is proposed that this is followed by 5-epimerisation by AvrF, O-methylation at C-3 catalysed by AvrH, and finally 4-ketoreduction by AvrE to produce dTDP-L-oleandrose. The same order of events has been proposed in the oleandomycin producer (Aguirrezbalaga et al., 2000) but more recently it has become clear that methylation in this case does not occur until the neutral sugar is attached to the macrolide aglycone (Rodriguez et al., 2001) so that the sugar actually transferred by the glycosyltransferase in the normal pathway is L-olivose.

As a result of such analyses, the use of plasmid systems to express deoxysugar biosynthetic pathways in a heterologous host is well known in the art. Using genes derived from the oleandomycin biosynthetic gene cluster in S. antibioticus, a plasmid (pOLV) containing a set of genes proposed to be required for the biosynthesis of dTDP-L-olivose and a second plasmid (pOLE) containing a set of genes proposed to be required for the biosynthesis of dTDP-L-oleandrose were constructed and introduced into a strain of Streptomyces albus which is not known to synthesise either these sugars or any macrolide polyketide, and which contains the oleG2 gene integrated into the genome. When supplied with the heterologous macrolide aglycone erythronolide B, 3-O-L-olivosyl erythronolide B was produced from the strain housing pOLV, and 3-O-L-oleandrosyl erythronolide B was produced from the strain housing pOLE. These results indicate that the gene sets carried by the respective plasmids are necessary and sufficient to produce the appropriate activated sugar substrate for the glycosyltransferase in a heterologous host strain (Aguirrezabalaga et al., 2000). In these plasmids the genes were largely in their natural gene order and expressed from their native promoters.

It has been shown more recently that the equivalent of these same eight genes from the avermectin biosynthetic pathway of S. avermitilis (avrBCDEFGHI) can be similarly expressed in a heterologous host under the control of their own promoters (Wohlert et al., 2001). They can also be expressed from a heterologous promoter, specifically that for the erythromycin resistance gene ermE* from S. erythraea. It was additionally shown that these oleandrose biosynthetic genes could be grouped into two distinct artificial operons, or expression cassettes, on a plasmid under the control of the overlapping divergent heterologous actI/actIII promoters from Streptomyces coelicolor, and that this led to the production of dTDP-oleandrose in Streptomyces lividans. Other versions of the expression plasmid were synthesised de novo in which individual oleandrose biosynthetic genes were eliminated from one cassette by in frame deletion; or omitted from the second cassette during construction. Avermectin aglycone was converted by S. lividans strains containing plasmids bearing such modified expression cassettes into glycosylated avermectin analogues. Thus, elimination of the gene avrF, the presumed 5-epimerase, led to the attachment of a D-sugar, D-olivose, to the aglycone. In other examples, mixtures of products were formed: for example, deletion of the presumed methyltransferase gene (avrH) led to glycosides with L-olivose or alternatively L-digitoxose (differing from L-olivose in having an inverted configuration at C-3) attached in place of oleandrose. The methylation was restored at low levels by complementing the latter strain with the 3-C-methyltransferase gene eryBIII to give so far uncharacterised avermectins. EryBIII operates within the biosynthetic pathway for the production of a closely related neutral deoxyhexose, mycarose, in the erythromycin producing strain of S. erythraea; eryBIII was introduced into the S. lividans host on a separate plasmid.

Despite these insights there remains considerable uncertainty over the identity and role of individual genes in the biosynthesis of many activated deoxy- and dideoxyhexoses. Further, there is considerable labour involved in the construction of the plasmid systems and great uncertainty over whether a particular combination of genes when cloned in a particular order will be properly expressed and will function as in their native context. Additionally, there is an urgent need for new methodologies for more rapid and convenient cassette-based expression of such sugar pathway genes, in particular in a manner that allows the rapid and flexible substitution, removal, addition, or rearrangement of the genes in the cassette in order to test and optimise the combinations of genes being used. In many of the experiments reported in the art, the degree of bioconversion was extremely limited and would not form the basis of any useful process for preparation of polyketide drugs. The reasons for this are unknown and may be complex, but it is obvious that one root of this problem could lie in the suboptimal performance of the various arbitrarily-constructed cassettes.

### SUMMARY OF THE INVENTION

The present invention shows that plasmid-based gene cassettes can be constructed which direct the synthesis of different dTDP sugars in a rationally designed and easy manner, with the sugar genes in each case flanked by unique restriction sites that facilitate gene exchanges. The design also allows the flexible and easy sequential linking of individual genes to build up the original gene cassette. The plasmid also has a unique XbaI site that can be used to co-clone additional genes with functions not present in the original plasmid. If such additional genes are cloned into the cassette plasmid using either SpeI, AvrII or NheI for the 5′-end and XbaI for the 3′-end of the gene then a unique XbaI restriction site is preserved for further use.

In the creation of the plasmid-based gene cassettes that are the subject of the invention, the genes may be cloned together from their natural context, as for example the genes for the initial three steps common in L-deoxysugar biosynthesis (dTDP-glucose synthase, oleS; 4,6-glucose dehydratase, oleE; 3,5-epimerase, oleL) (FIG. 2) which were cloned on a single fragment in the example of cassette plasmid pLN2 described below. Alternatively and preferably, they may be individually generated by PCR and flanked by unique restriction sites introduced by the PCR process. Each amplified gene contains its own ribosomal binding site (Table 2). Two of the unique restriction sites (HindIII and XbaI) are used to facilitate subcloning of each amplicon in E. coli vectors (eg. pUC 18) in order to generate a sugar gene library. The other two restriction sites are used to facilitate the sequential addition of sugar genes in order to generate the initial plasmid construct. Also, these restriction sites allow exchange of genes with similar functions in order either to confirm function, or to assay the substrate flexibility of the different enzymes. These restriction sites were selected since they cut very rarely in DNA of high G+C content typical of actinomycetes. Thus, for example, the 4-ketoreductase gene oleU can be replaced by one of a number of other natural 4-ketoreductase genes, which may subsequently lead to the formation of different product activated sugars (FIG. 8). In one of the examples described here the use of the gene eryBIV leads to the production of the unusual sugar L-digitoxose (FIG. 9), with its subsequent attachment to an aglycone, in good yield. If the basic cassette, for example the plasmid pLN2 (FIGS. 3A, 4, and 6) whose construction is given below, directs the biosynthesis of 2,6-dideoxysugars, then to generate cassette plasmids for 6-deoxysugars, the two genes involved in the 2-deoxygenation process in sugar biosynthesis (oleV and oleW) can be easily removed. Specifically, for example using the two unique restriction sites (AvrII and SpeI) flanking this pair of genes (FIGS. 6 & 11), a plasmid pLN2Δ can be generated which is competent for the biosynthesis of L-rhamnose.

Using such plasmid-based gene cassettes, glycosyltransferase enzymes can be rapidly screened for their ability to attach a range of activated sugars to a range of exogenously supplied, or endogenously generated, aglycone templates. Patent application WO 01/79520 for example demonstrates that such glycosyltransferases frequently show surprising flexibility towards both aglycone and sugar substrates, and that this process allows the production of novel glycosylated polyketides in good yield. This overcomes the problem not only of supplying novel sugar attachments to individual polyketides, including polyketides altered by genetic engineering, but also of increasing the diversity of polyketide libraries by combinatorial attachment of sugars. It is particularly surprising that new glycosylated products can be produced in systems in which one or more of the components are heterologous to each other, the components being selected from the aglycone template, the sugar moiety or moieties, the glycosyltransferase, the host cell and/or genes encoding enzymes capable of modifying the sugar moiety, either before or after attachment to the aglycone template. In preferred embodiments, two, three, four, or all of the components are heterologous to each other.

It will readily occur to those skilled in the art that the methods described here can be readily extended to embrace alternative sugar genes from other natural sugar biosynthetic pathways. Examples of such pathways, for which detailed information is already available, include the sugars desosamine, mycarose, mycaminose, rhodinose, oliose, olivose and oleandrose. There is also the opportunity to select the nature of the activated sugar to be either in the L- or in the D- form. The cassette arrangement for several of these further sugars is shown in FIGS. 13 and 14.

It will be additionally readily apparent that even in those cases where the initial screening for the presence of the predicted activated sugar product did not show its transfer to the provided aglycone, this may simply reflect the inability of the glycosyltransferase to catalyse that particular transformation, rather than the non-functionality of the sugar cassette itself. Therefore, as the lexicon of glycosyltransferases is expanded, and as knowledge increases about their range of substrate specificity, so the useful range of plasmid-based gene cassettes encoding the biosynthesis of deoxyhexose (sugar) templates constructed as described here will correspondingly increase.

Accordingly, in a first aspect, the present invention provides a process for producing a hybrid glycosylated product by transferring one or more sugar moieties to an aglycone template, the process comprising:
transformation of microorganism host cells with nucleic acid encoding a plasmid-based gene cassette which contains the genes sufficient to direct the synthesis of a specific activated sugar in those host cells; and also with nucleic acid encoding a glycosyltransferase (GT); and,
providing an aglycone template to the transformed microorganism so that the GT transfers one or more sugar moieties to the aglycone template in order to produce a hybrid glycosylated product;
wherein one or more of the sugar moieties, the aglycone template, the glycosyltransferase, the genes encoding enzymes capable of modifying sugar moieties or the host cells are heterologous to the other components of the invention.

In a further aspect, the present invention provides host cells transformed with nucleic acid encoding a gene expression cassette which contains the genes sufficient to direct the synthesis of a specific activated sugar in those host cells, optionally operably linked under the same promoter; and also transformed with a glycosyltransferase (GT), wherein the GT is heterologous to the host cells and transfers one or more sugar moieties to an aglycone template within the cells to produce a hybrid glycosylated product. Examples of specific activated deoxysugars include, but are limited to the following: L-NDP-olivose, L-NDP-oleandrose, L-NDP-oliose, L-NDP-mycarose, L-NDP-cladinose, L-NDP-digitoxose, L-NDP-3-O-methyldigitoxose, L-NDP-rhamnose, L-NDP-2-O-methylrhamnose, L-NDP-3-O-methylrhamnose, L-NDP4-O-methylrhamnose, L-NDP-2,3-di-O-methylrhamnose, L-NDP-2,3,4-tri-O-methylrhamnose, L-NDP-rhodinose

In a further aspect, the present invention provides a process for producing a hybrid glycosylated product, the process comprising culturing the host cells defined above and isolating the product thus produced. In embodiments in which the aglycone template is supplied to the host cells, rather than being produced by the host cell, the process may comprise the additional step of supplying the aglycone template to the cells.

In further aspects, the present invention provides hybrid glycosylation products as obtainable by any of the processes disclosed herein.

Embodiments of the present invention will now be described by way of example, and not limitation, with reference to the accompanying figures.

### BRIEF DESCRIPTION OF THE FIGURES

FIG. 1: Structure of oleandomycin.

FIG. 2: Organisation of the genes required for dTDP-L-oleandrose biosynthesis from the gene cluster of Streptomyces antibioticus governing the biosynthesis of oleandomycin.

FIG. 3: Scheme for the PCR amplification and cloning of individual genes encoding enzymes that catalyse steps in deoxysugar biosynthesis to create sugar gene libraries, and their incorporation stepwise into an expression cassette plasmid.

FIG. 4: Detailed scheme for construction of plasmid pLN1.

FIG. 5: Scheme showing the TLC based screening of plasmid pLN1 housed in Streptomyces albus GB16 for its ability to produce activated L-olivose which is then attached to erythronolide B in the presence of the glycosyltransferase OleG2. Because the methyltransferase OleY is also present the attached sugar is subsequently methylated to provide oleandrosyl-erythronolide B.

FIG. 6: Scheme showing the derivation of expression cassette plasmid pLN2 in which the XbaI site is now unique; and the scheme for extrusion of oleV and oleW genes to create expression cassette plasmid pLN2VW which provides for synthesis of dTDP-L-rhamnose.

FIG. 7: Schemes showing the methods used to test for the intracellular production of activated sugars, through the catalysis of their attachment to suitable aglycones in the presence of a suitable glycosyltransferase. The glycosyltransferase ElmGT for example is known to transfer L-rhamnose, L-olivose, L-rhodinose, D-olivose and D-mycarose to the aglycone 8-demethyltetracenomycin C.

FIG. 8: Scheme showing the exchange of a heterologous 4-ketoreductase gene for the oleU 4-ketoreductase gene in plasmid pLN2.

FIG. 9: Scheme showing the natural substrate for 4-ketoreductase EryBIV and alternative substrates for this enzyme.

FIG. 10: Products of the fermentation of strain LI16 containing plasmid pLN2EryBIV.

FIG. 10A: HPLC analysis of the production of glycosylated products in the strain NAG2 (containing the glycosyltransferase OleG2, as well as the plasmid pLN2EryBIV, and supplied with Erythronolide B.

FIG. 11: Scheme showing the deletion of genes oleV and oleW from plasmid pLN2.

FIG. 12: Products containing L-rhamnose from the fermentation of strains containing plasmid pLN2ΔVW.

FIG. 12A: production of glycosylated products in the strain S. albus NAG2 (containing the glycosyltransferase OleG2, as well as the plasmid pLN2ΔVW, and supplied with erythronolide B.

FIG. 13: Scheme showing the arrangement of genes in the expression cassette plasmid pDES.

FIG. 14: Construction of expression cassette plasmids for the production of dTDP-L-olivose and dTDP-L-mycarose.

FIG. 15: Shows the arrangement of genes in the expression plasmid PKS.

FIGS. 16 and 17 are diagrams for use in describing and explaining the construction of the plasmids described in this patent.

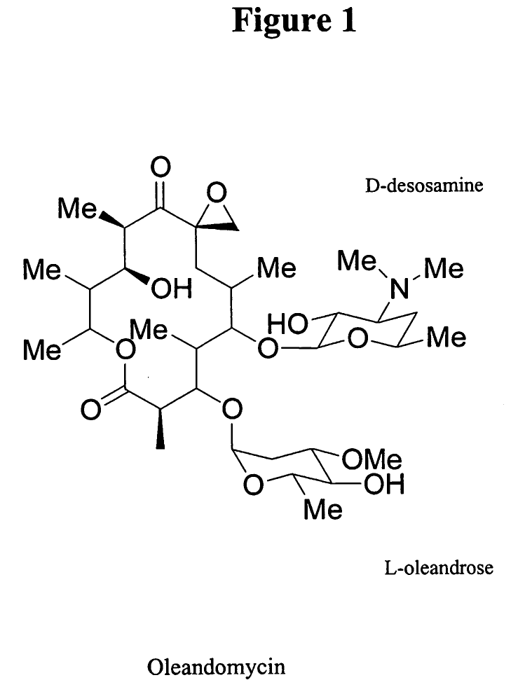

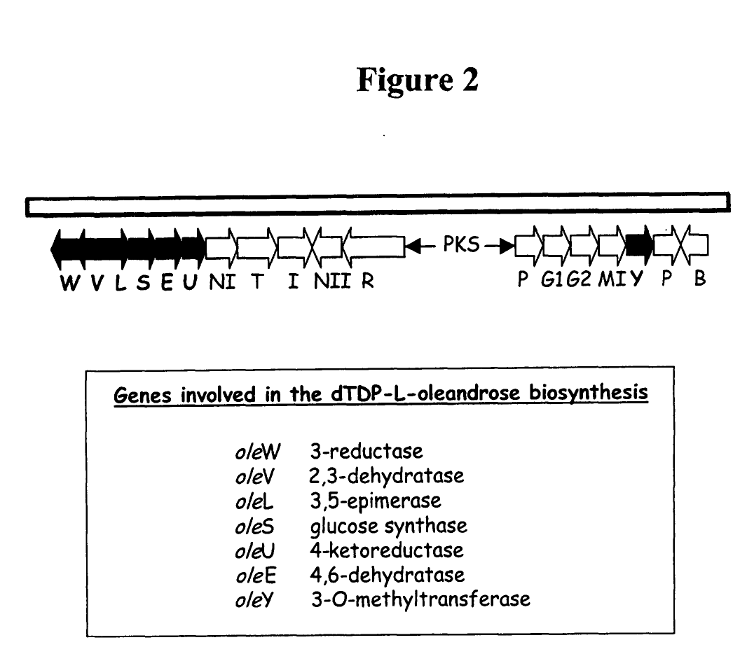

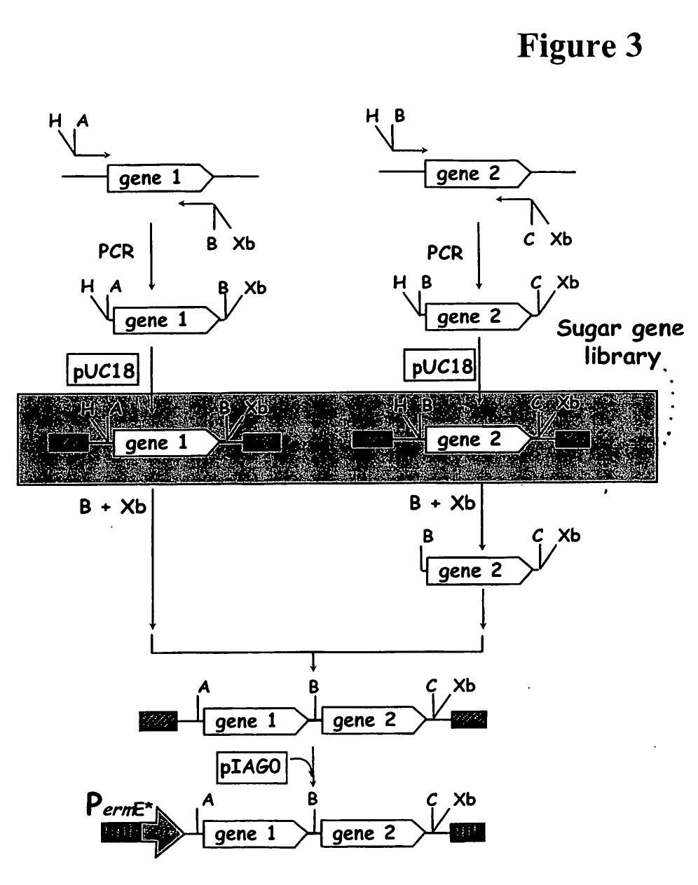

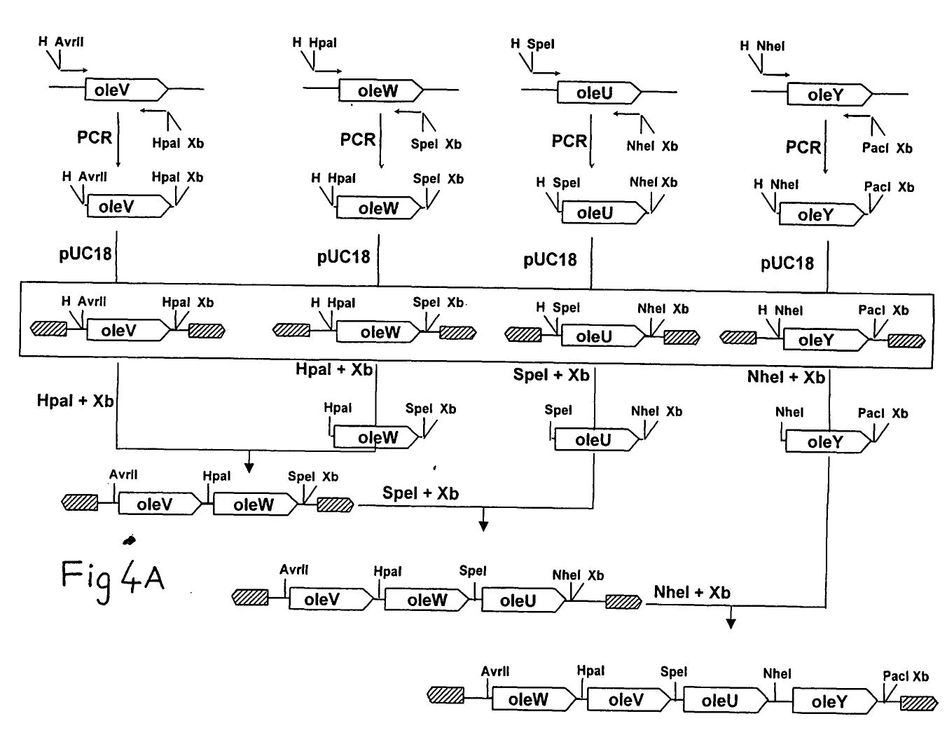

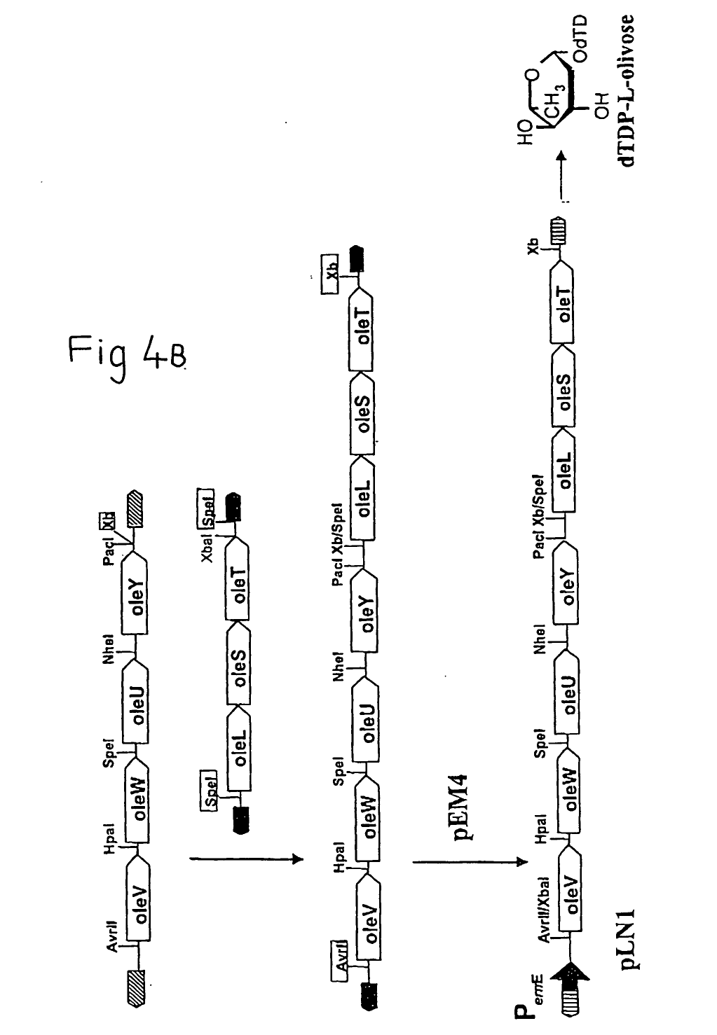

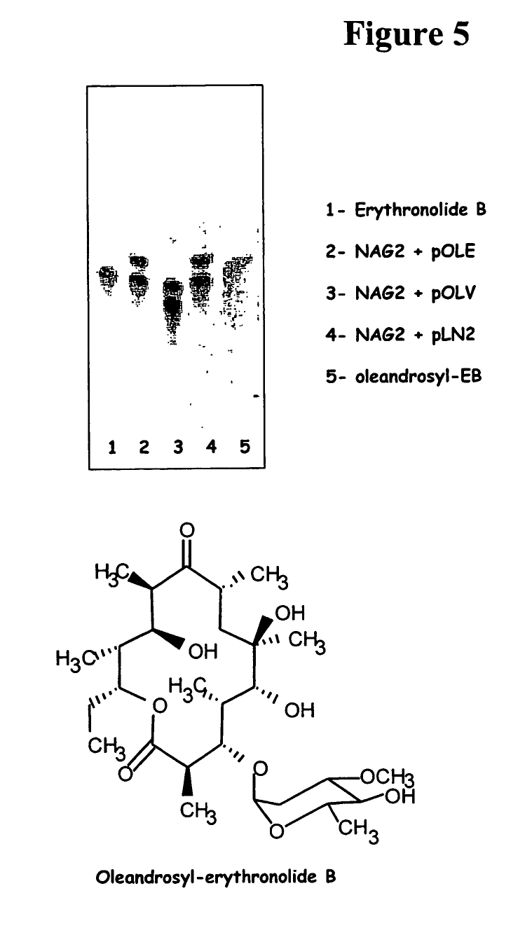

Table 1: Genes used in the construction of gene cassette plasmids.

Table 2: Synthetic oligonucleotides used in the PCR amplification of individual deoxysugar pathway genes for construction of pathway expression gene cassettes. Each oligonucleotide contains both general and specific restriction sites, and the forward primers contain the requisite ribosomal binding site motif and start codon.

Table 3: Plasmid constructs directing the biosynthesis of different sugars and glycosyltransferase (GTF) systems used to show the presence of the sugar.

Methods and Materials

Microorganisms, Culture Conditions and Vectors

Streptomyces antibioticus ATCC11891 (oleandomycin producer), Streptomyces fradiae ATCC 19609 (tylosin producer), Streptomyces peucetius ATCC29050 (daunorubicin producer), Streptomyces nogalater NRRL3035 (nogalamycin producer), and Saccharopolyspora erythraea NRRL2338 (erythromycin producer) were used as source of DNA. Streptomyces albus GB16 (Blanco et al., 2001) was used as host for gene expression and for biotransformation experiments. Streptomyces argillaceus 16F4 (Blanco et al., 2001) was used for obtaining 8-demethyl-tetracenomycin C (8DMTC). Bacterial growth was carried out on trypticase soya broth (TSB; Oxoid) or R5A medium (Fernandez et al., 1998). To obtain spores growth was carried out for 7 days at 30° C. on agar plates containing A-medium (Fernández et al., 1998). Escherichia coli XL1-Blue (Bullock et al., 1987) was used as a host for subcloning and was grown at 37° C. in TSB medium. pLITMUS29 (Biolabs) and pUC18 were used as vectors for subcloning experiments and DNA sequencing. pWHM3 (Vara et al., 1989) and pEM4 (Quirós et al., 1998) were used for expression in Streptomyces. Where antibiotic selection was required thiostrepton (25 μg/ml), apramycin (25 μg/ml) or ampicillin (100 μg/ml) were used.

DNA Manipulation and Sequencing

Plasmid DNA preparations, restriction endonuclease digestions, alkaline phosphatase treatments, ligations and other DNA manipulations were performed according to standard procedures for E. coli (Sambrook et al., 1989) and for Streptomyces (Kieser et al., 2000). DNA sequencing was performed using the dideoxynucleotide chain-termination method (Sanger et al., 1977) and the Cy5 AutoCycle Sequencing Kit (Pharmacia Biotech). Both DNA strands were sequenced with primers supplied in the kits or with internal oligoprimers (17-mer) using an ALF-express automatic DNA sequencer (Pharmacia). Computer-assisted data base searching and sequence analyses were carried out using the University of Wisconsin Genetics Computer Group programs package (UWGCG; Devereux et al., 1984) and the BLASTP program (Altschul et al., 1990).

PCR Amplification of the Genes

Individual genes were amplified by PCR using the oligonucleotide primers listed in Table 2. These primers were designed to create HindIII and XbaI sites at the 5′-end and 3′-end respectively of all genes in order to facilitate subcloning. Moreover, two other restriction sites were included in each pair of oligoprimers which were specific for each gene, to facilitate the exchange of specific genes. PCR reaction conditions were as follows: 100 ng of template DNA were mixed with 30 pmol of each primer and 2 units of Vent DNA Polymerase (New England Biolabs) in a total reaction volume of 50 μl containing 2mM of each dNTP, 10 mM KCl, 10 mM (NH₄)₂SO₄, 20 mM Tris-HCI (pH 8.8), 2 mM MgSO₄, 0.1% Triton X-100 and 10% DMSO (Merck). This reaction mix was overlaid with 50 μl of mineral oil (Sigma) and the polymerization reactions were performed in a thermocycler (MinyCycler, MJ Research). The PCR products were purified and subcloned into pUC18.

Construction of Cassette Plasmids

Two gene cassette plasmids were initially constructed:

Plasmid pLN1: for constructing this plasmid, the oleandomycin sugar genes, oleV, oleW, oleU and oleY, were independently amplified using the oligoprimers described in Table 1 and the conditions above. Subsequently, all the genes were sequentially cloned into pUC18 to generate pUC 18VWUY. To achieve this, each gene was subcloned using the 5′specific restriction site and the XbaI site (located at 3′-end of each gene) into intermediate plasmid constructs which were digested with the same restriction enzymes. Then, the AvrII- XbaI fragment containing the four genes was cloned downstream of the erythromycin resistance promoter of pEM4, generating pLN. Finally, a SpeI-XbaI fragment (using these sites from the polylinker) containing oleL, oleS and oleE was subcloned from pLR234Δ7 (Rodriguez et al., 2000) into the XbaI site of pLN, generating pLN1.

Plasmid pLN2. First, the oleV PCR fragment was subcloned as an AvrII-XbaI fragment into the same sites of pLITMUS29 and rescued as a SpeI (using this site from the polylinker)-XbaI fragment for subcloning into the XbaI site of pEM4, downstream of the erythromycin resistance promoter, generating pEM4V. Then, oleV in pLN1 was exchanged by oleV from pEM4V generating pLN1b, by digesting both constructs with HindIII and HpaI. Finally the HindIII-XbaI fragment of pLN1b that contains all of the oleandrose biosynthetic genes from S. antibioticus under the control of the erythromycin resistance promoter, was subcloned into the same sites of pWHM3, generating plasmid pLN2.

Various derivatives of plasmid pLN2 were made as follows:

Plasmid pLN2ΔVW: Plasmid pLN2 was digested with AvrII and SpeI and the larger of the two DNA fragments produced was purified by gel electrophoresis and then ligated to itself (as AvrII and SpeI have compatible ends) to obtain the desired plasmid pLN2ΔVW.

Plasmids pLN2 EryBIV, pLNSnogC, pLNDnmV, pLNTylD: Plasmid pLN2 was digested with SpeI and NheI and the larger of the two DNA fragments produced was isolated. The eryBIV gene was amplified by PCR using the oligonucleotide primers 5′-B4U and 3′-B4L listed in Table 2, and cloned into pUC18. The eryBIV gene insert was excised from this clone and ligated with the SpeI-NheI digested pLN2 backbone to create plasmid pLN2EryBIV. Similarly, the snogC gene from S. nogalater, the dnmV gene of S. peucetius, and the tylD gene of S. fradiae were each amplified using the appropriate forward and reverse oligonucleotide primers listed in Table 2, and cloned into pUC18. The respective gene inserts were excised from these clones and ligated into the SpeI-NheI digested backbone of pLN2 to create plasmids pLN2SnogC, pLN2DnmV and pLN2TylD respectively.

Transformation of Biotransformation Host Cells

The plasmid based gene cassettes and glycosyltransferases were introduced into the two biotransformation host organisms S. lividans TK21 and S. albus GB16 by protoplast transformation according to the standard procedures (Kieser et al., 2000).

Biotransformation and Chromatographic Techniques

Spores of the appropriate S. albus GGB16 or S. lividans TK24 recombinant strains carrying the relevant GT and plasmid-based gene cassette for deoxysugar biosynthesis were grown in the presence of 8-demethyl-tetracenomycin C. (Blanco et al., 2001) or erythronolide B (Aguirrezabalaga et al., 2000) according to conditions previously described. TLC, HPLC and LCMS analyses were performed as previously described (Aguirrezabalaga et al., 2000; Fernández-Lozano et al., 2000; Blanco et al., 2001).

Specific Examples of Products Identified from Engineered Biotransformation Strains

### EXAMPLE 1

### Glycosylation Reporter Systems

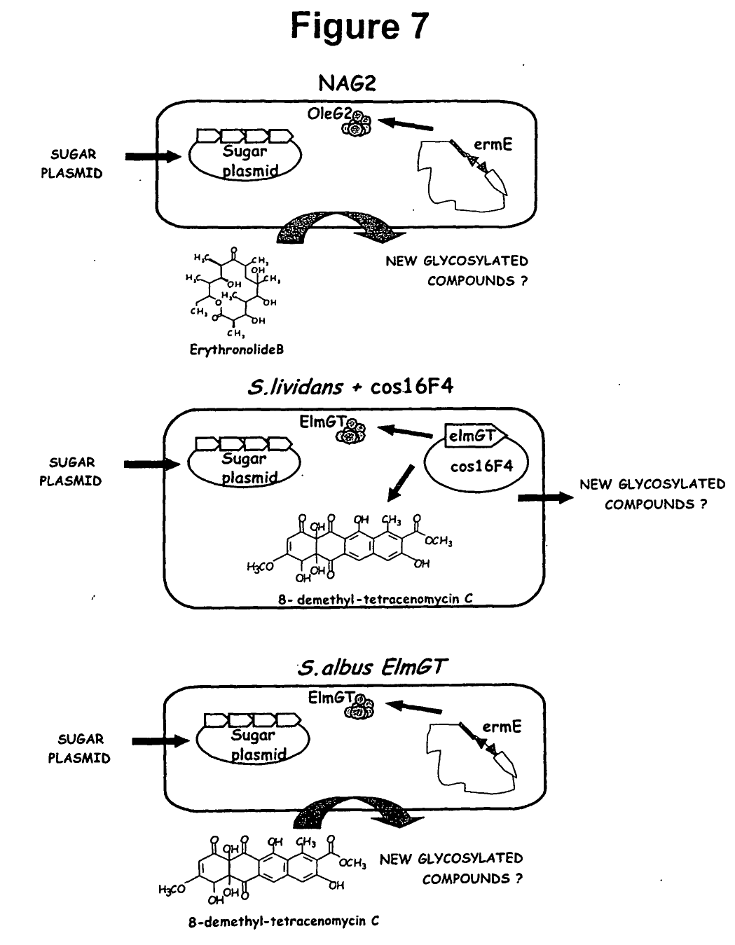

The glycosyltransferase, ElmGT, of the elloramycin pathway, has broad substrate specificity towards several L-6-deoxysugars (L-olivose, L-rhamnose, L-rhodinose) and D-6-deoxysugars (D-olivose, D-mycorose). ElmGT is capable of transferring such sugars onto the aglycone 8-demethyl-tetracenomycin C (8DMTC) (FIG. 7). The glycosyltransferase, OleG2, of the oleandomycin pathway, has broad substrate specificity towards several 6-deoxysugars. OleG2 is capable of transferring such sugars onto the aglycone erythronolide B (EB) (FIG. 7). The glycosyltransferase, EryBV, of the erythromycin pathway, has broad substrate specificity towards several 6-deoxysugars. EryBV is capable of transferring such sugars onto the aglycone EB (FIG. 7).

a. Provision of endogenous 8DMTC in S. albus.

S. albus 16F4, a recombinant strain harbouring cosmid 16F4 that directs endogenous biosynthesis of 8DMTC and contains elmGT, provides a reporter strain that when transformed with pLN2 derivatives allows the detection of plasmid-directed synthesis of NDP-6-deoxysugars by their transfer to 8DMTC and the subsequent formation of the corresponding glycosylated compound (FIG. 7).

b. Provision of endogenous 8DMTC in S. lividans.

S. lividans LI16, a recombinant derivative strain of S. lividans TK21 harbouring cosmid 16F4 that directs endogenous biosynthesis of 8DMTC and contains elmGT, provides a reporter strain that when transformed with pLN2 derivatives allows the detection of plasmid-directed synthesis of NDP-6-deoxysugars by their transfer to 8DMTC and the subsequent formation of the corresponding glycosylated compound (FIG. 7).

c. S. albus GB16, a biotransformation strain for 8DMTC
S. albus GB16, a recombinant strain with elmGT integrated into the chromosome and expressed under the control of ermE* (the erythromycin resistance promoter of S. erythraea), provides a reporter strain that when transformed with pLN2 derivatives and fed the aglycone 8DMTC allows the detection of plasmiddirected synthesis of NDP-6-deoxysugars by their transfer to 8DMTC and the subsequent formation of the corresponding glycosylated compound (FIG. 7).

d. S. lividans NAG2, a biotransformation strain for 8DMTC or erythronolide B.
S. lividans NAG2, a recombinant strain with oleG2 integrated into the chromosome and expressed under the control of ermE* (the erythromycin resistance promoter of S. erythraea), provides a reporter strain that when transformed with pLN2 derivatives and fed the aglycone 8DMTC or erythronolide B (EB) allows the detection of plasmid-directed synthesis of NDP-6-deoxysugars by their transfer to 8DMTC or EB and the subsequent formation of the corresponding glycosylated compound (FIG. 7).

e. S. lividans NAB5, a biotransformation strain for 8DMTC or erythronolide B.
S. lividans NAB5, a recombinant strain with eryBV integrated into the chromosome and expressed under the control of ermE* (the erythromycin resistance promoter of S. erythraea), provides a reporter strain that when transformed with pLN2 derivatives and fed the aglycone 8DMTC or erythronolide B (EB) allows the detection of plasmid-directed synthesis of NDP-6-deoxysugars by their transfer to 8DMTC or EB and the subsequent formation of the corresponding glycosylated compound.

### EXAMPLE 2

### Functionality of pLN2

S. albus 16F4, S. albus GB16 and S. lividans NAG2 were transformed with pLN2 encoding the biosynthetic pathway for the production of L-olivose (see Materials and Methods above). S. albus 16F4+pLN2 was found to produce a compound consistent with L-olivosyl-tetracenomycin C (LOLV-TCMC) in terms of HPLC mobility and absorption spectrum when compared with the pure compound used as standard (Table 3). S. albus GB16+pLN2, when fed with 8DMTC, converted the aglycone to a compound consistent with LOVL-TCMC in terms of HPLC mobility and absorption spectrum when compared with the pure compound used as standard (Table 3). MALDI-TOF analysis of this compound showed molecular peaks at m/z 611.0815 and 627.0728 for the sodium and potassium adducts of LOVL-TCMC, respectively (Table 3). The reporter system providing endogenous 8DMTC (S. albus 16F4) showed a greater efficiency of conversion to LOVL-TCMC (64%) compared to the biotransformation system (S. albus GB16) with only 12% conversion of the aglycone observed.

S. lividans NAG2+pLN2 was found to convert 50% of supplied EB to a compound with HPLC mobility, absorption spectrum and LCMS analysis consistent with L-oleandrosyl-erythronolide B (LOLE-EB) as compared with a pure standard.

S. lividans NABV+pLN2 was found to convert supplied EB to a compound with HPLC mobility, absorption spectrum and LCMS analysis consistent with L-oleandrosyl-erythronolide B (LOLE-EB) as compared with a pure standard.

### EXAMPLE 3

### Screening of Heterologous 4-ketoreductases in pLN2 Acting on L-6-deoxysugar Intermediates

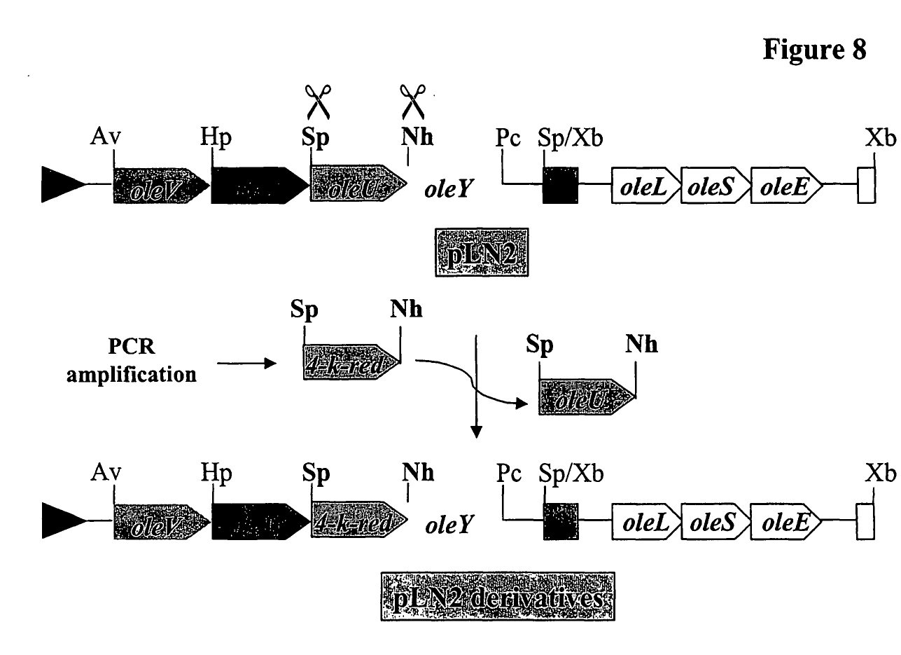

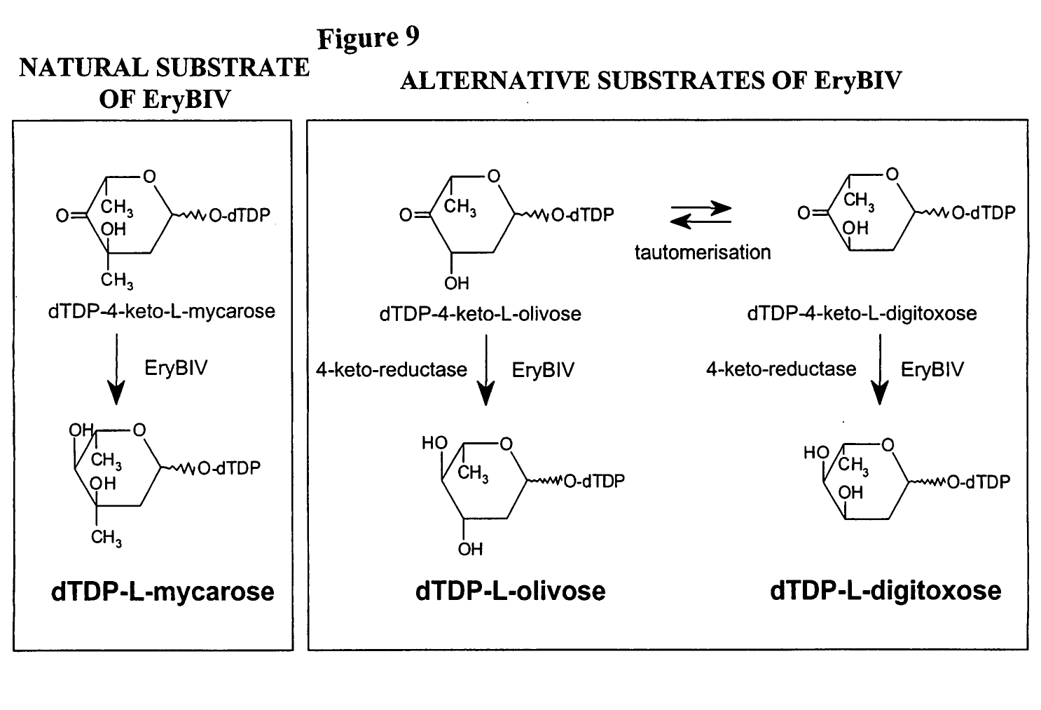

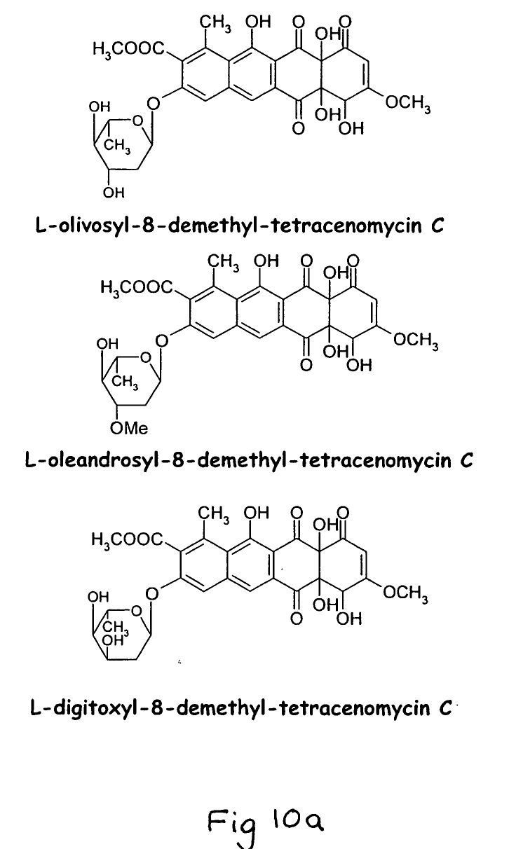

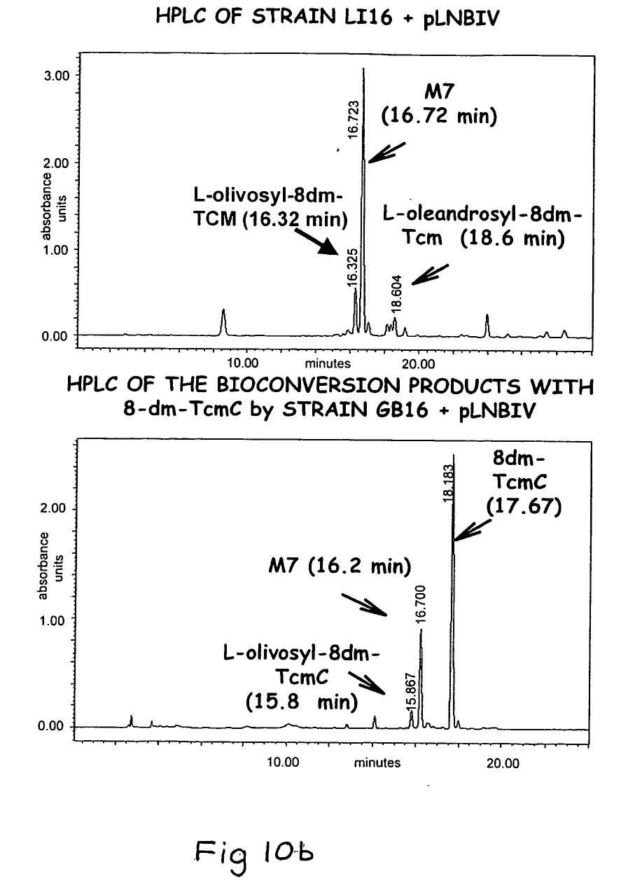

The 4-ketoreductase encoded by oleU was replaced, as described in the Material and Methods above and FIG. 8, with one of the following heterologous 4-ketoreductases eryBIV or snogC of the erythromycin and nogalamycin clusters, respectively (Table 1). These reductases act on different sugar intermediates to OleU, but share the same C4 ketoreductase stereospecificity.

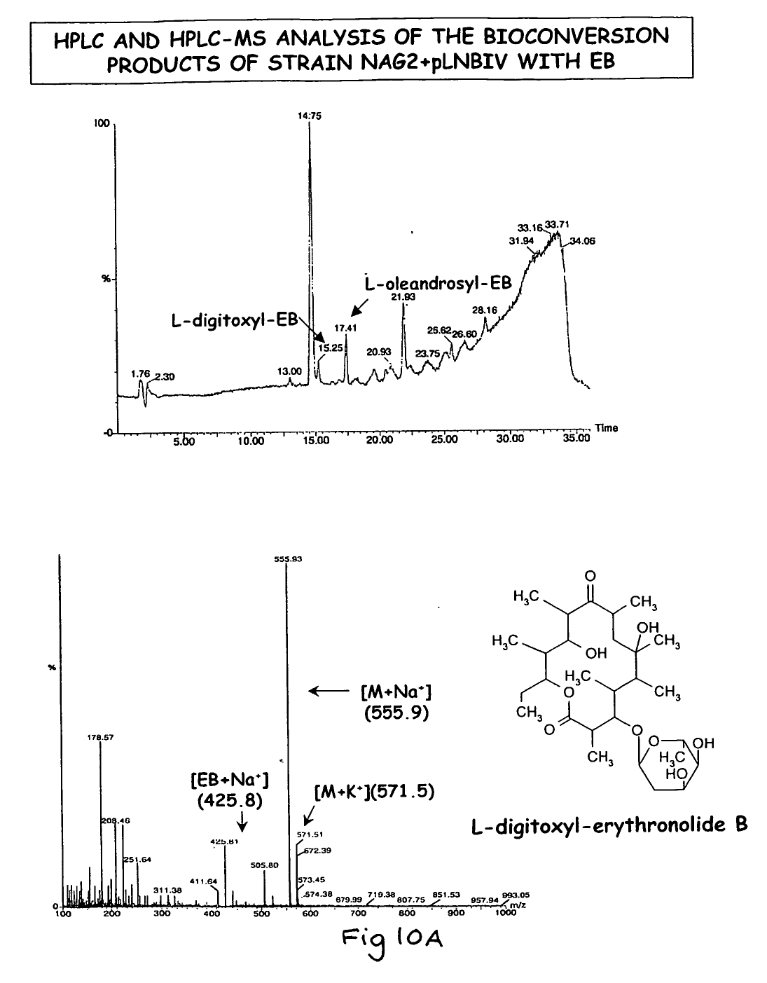

Replacement of oleU with eryBIV (pLN2 derivative pLNBIV) gave rise to a new HPLC peak in both reporter systems S. albus 16F4 and S. albus GB16 (described in Example 1), with the conversion of 8DMTC at levels of 20% and 3% respectively. This new compound was consistent with LOLV-TCMC in terms of HPLC mobility and absorption spectrum when compared with the pure compound used as standard (Table 3). MALDI-TOF analysis of this compound showed molecular peaks at m/z 611.0815 and 627.0728 for the sodium and potassium adducts of LOLV-TCMC, respectively (FIG. 10).

No glycosylation of 8DMTC was observed on replacement of oleU with snogC (pLN2 derivative pLNS). The expected products of SnogC, L-olivose or L-rhamnose, are readily transferred to 8DMTC by ElmGT, lack of glycosylation indicates a narrow sugar substrate specificity of the snogC gene product.

Replacement of oleU with eryBIV (pLN2 derivative pLNBIV) gave rise to two new HPLC peaks in the reporter system S. lividans NAG2 fed with EB. These new compounds were consistent with a minor peak of LOLE-EB and a major peak of L-digitoxosyl-EB (LDIG-EB) in terms of HPLC mobility and absorption spectrum when compared with the pure compound used as standard. LCMS analysis of LDIG-EB showed molecular peaks at m/z 555.0 and 571.5 for the sodium and potassium adducts, respectively (FIG. 10A).

Replacement of oleU with eryBIV (pLN2 derivative pLNBIV) gave rise to three new HPLC peaks in the reporter system S. lividans NAB5 fed with EB. These new compounds were consistent with two minor peaks corresponding to LOLE-EB and L-3-methyl-digitoxosyl-EB (L3MDIG-EB) and a major peak of L-digitoxosyl-EB (LDIG-EB) in terms of HPLC mobility, absorption spectrum and LCMS analysis when compared with the pure compound used as standard.

### EXAMPLE 4

### Screening of Heterologous 4-ketoreductases in pLN2 Acting on D-6-deoxysugar Intermediates

The 4-ketoreductase TylD acts on D-6deoxysugar intermediates but has the same stereospecificity at C-4 as OleU (Table 1). No glycosylation of 8DMTC was observed on replacement of oleU with tylD (pLN2 derivative pLNT) in the S. albus or S. lividans host strain reporter systems.

### EXAMPLE 5

### Biosynthesis of L-rhamnose by Two-gene Deletion of oleV and oleW (pLN2 Derivative pLN2Δ)

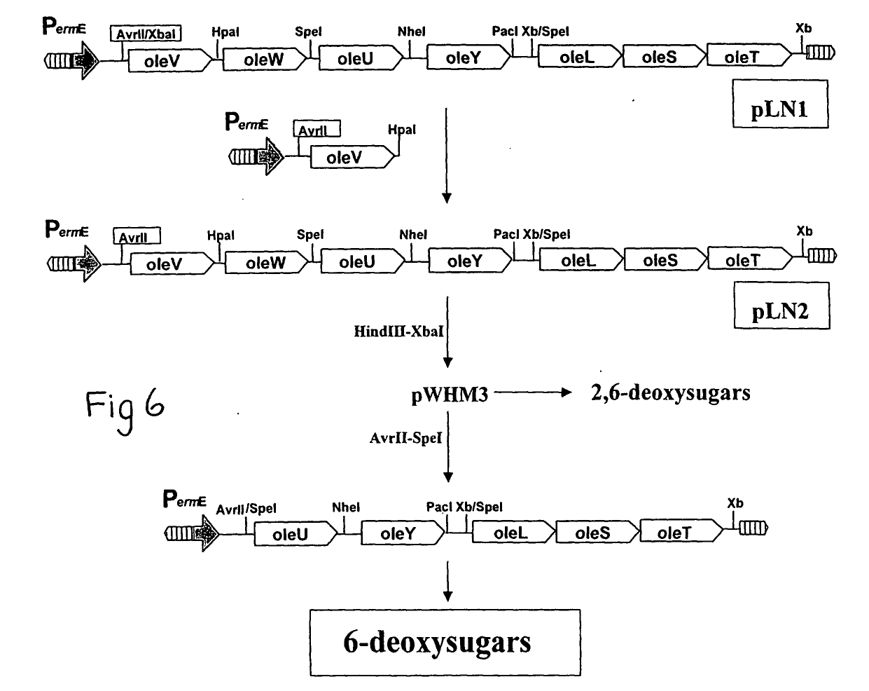

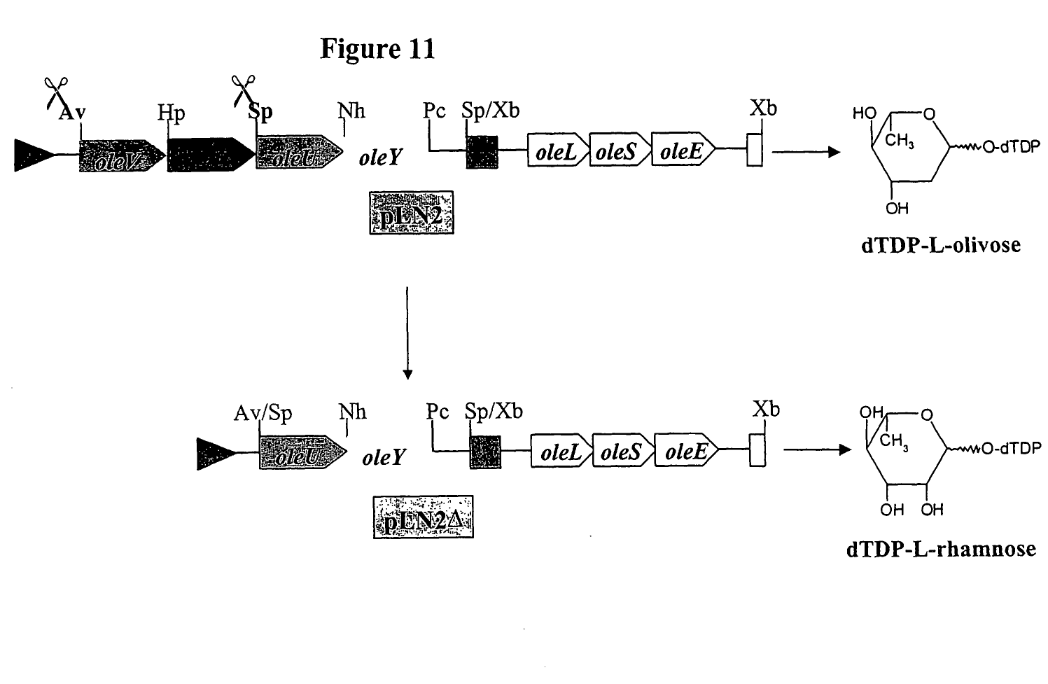

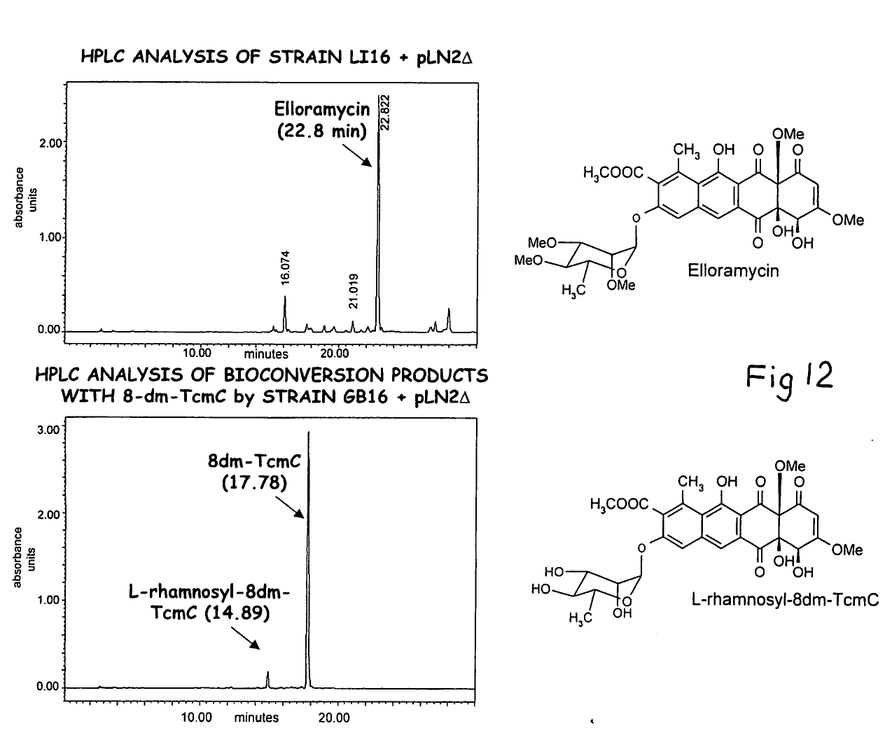

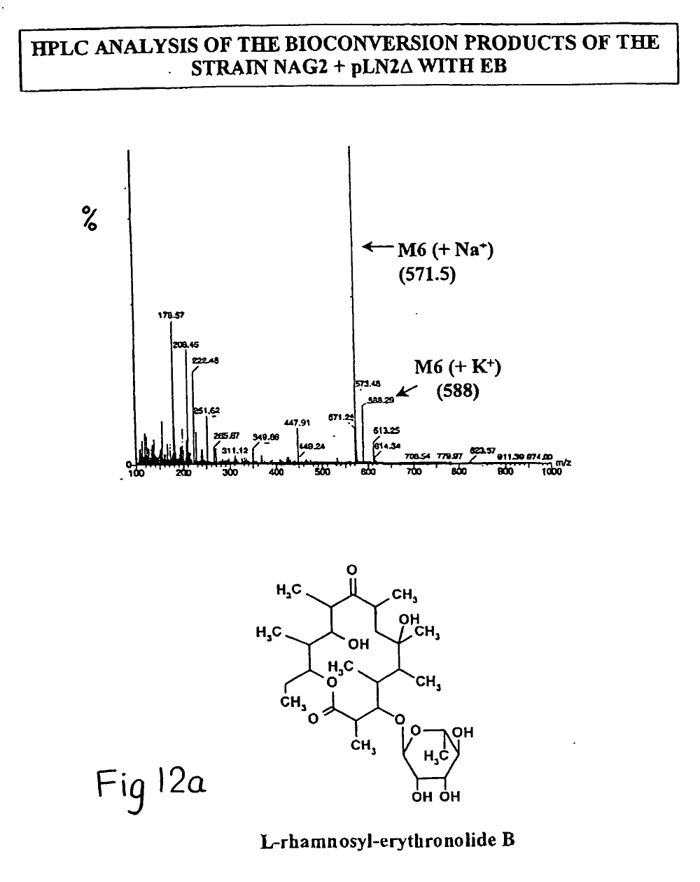

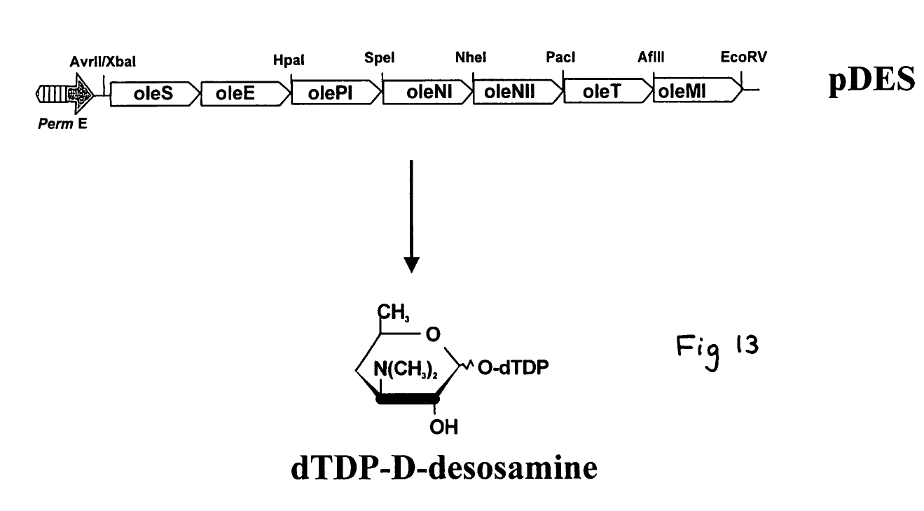

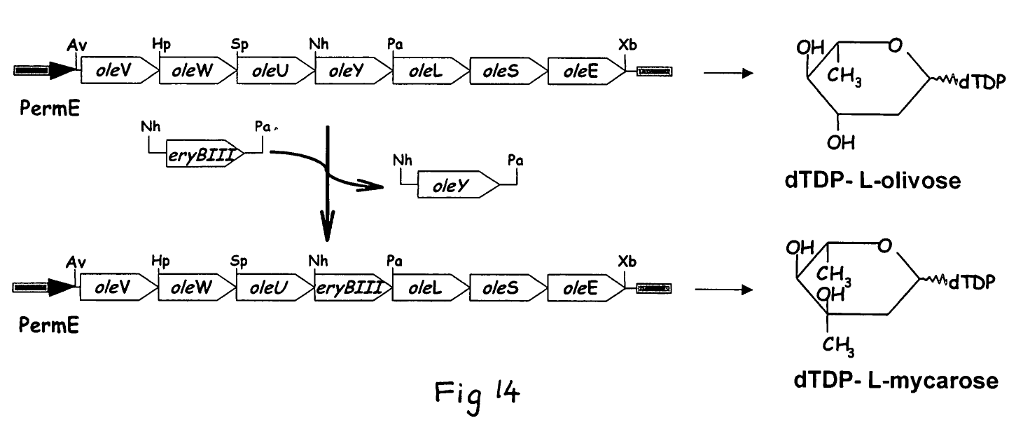

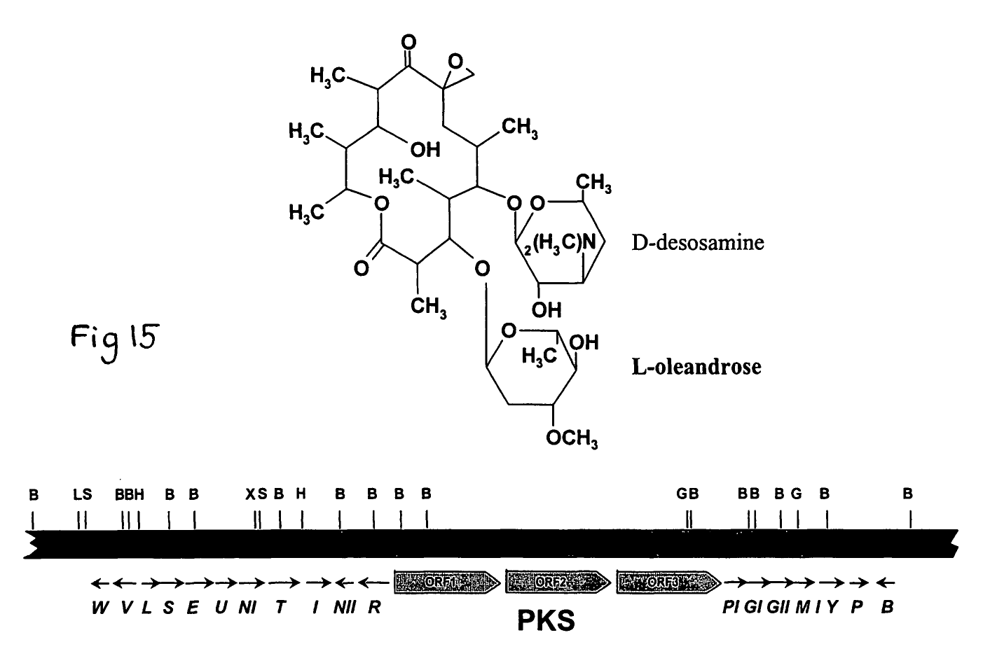

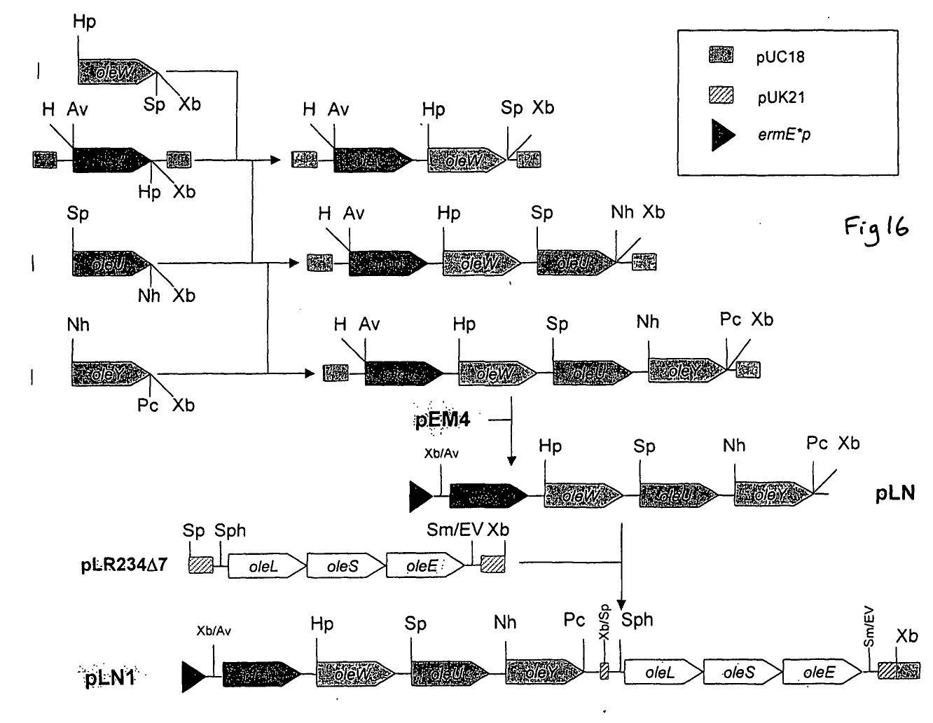

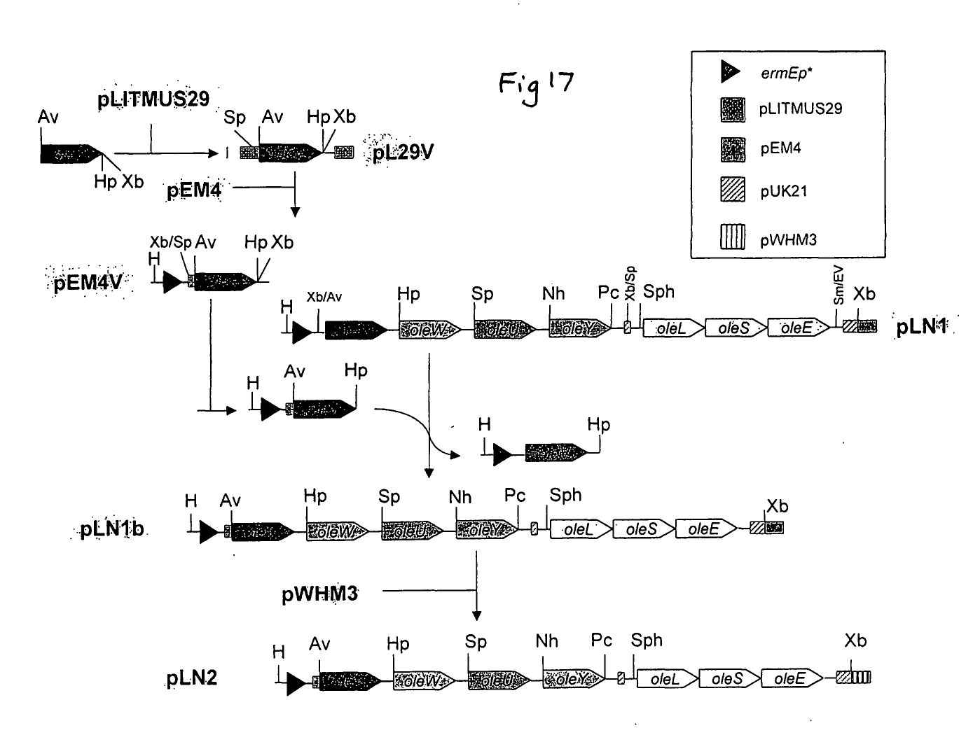

L-Rhamnose differs from L-olivose in containing an hydroxyl group at C-2. The cassette design of pLN2 enables the removal of the deoxygenation genes oleV and oleW, creating a pLN2 derivative pLN2Δ. The genes oleV and oleW were removed by digestion of pLN2 with AvrII and SpeI (generating compatible cohesive ends) and further re-ligation. The resulting construct should direct the biosynthesis of L-rhamnose (FIG. 6 and FIG. 11). The construct, pLN2Δ, gave rise to a new glycosylated derivative in both reporter systems S. albus 16F4 and S. albus GB16 (described in Example 1). Once ElmGT transferred L-rhamnose onto the aglycone 8DMTC, the three O-methyltransferases present in cos16F4 methylated the three free hydroxyl groups of L-rhamnose with the formation of elloramycin in S. albus 16F4 (FIG. 12). This new compound was consistent with elloramycin in terms of HPLC mobility and absorption spectrum when compared with the pure compound used as standard. MALDI-TOF analysis, in the negative mode, of this compound showed a molecular peak at m/z 659.0380, corresponding to elloramycin. In S. albus GB16, MALDI-TOF analysis showed molecular peaks at m/z 627.0641 and 643.0388, corresponding to the respective sodium and potassium adducts of L-rhamnose-tetracenamycin C (LRHA-TCMC). The construct, pLN2Δ, gave rise to a new glycosylated derivative in the reporter system S. lividans NAG2. This new compound was consistent with L-rhamnosyl-EB (LRHA-EB) in terms of HPLC mobility and absorption spectrum when compared with the pure compound used as standard. LCMSanalysis of this compound showed molecular peaks at m/z 571.5 and 588.0, corresponding to the sodium and potassium adducts of LRHA-EB (FIG. 12A).

### EXAMPLE 6

### Biosynthesis of L-rhamnose by Two-gene Deletion of oleV and oleW (pLNBIV Derivative pLNBIVΔ)

L-Rhamnose differs from L-olivose in containing an hydroxyl group at C-2. The cassette design of pLNBIV enables the removal of the deoxygenation genes oleV and oleW, creating a pLNBIV derivative pLNBIVΔ. The resulting construct should direct the biosynthesis of L-rhamnose (FIG. 6).

No glycosylated products were formed, demonstrating that EryBIV requires a C-2-deoxy sugar intermediate, whereas OleU has a broader substrate specificity acting on both 2,6-dideoxy and C-2-deoxy sugar intermediates (Example 3).

### EXAMPLE 7

### Biosynthesis of L-rhodinose (pLN2 Derivative pLNRHO)

L-olivose (2,6-dideoxysugar) and L-rhodinose (2,3,6-trideoxysugar with an equatorial hydroxyl group at C-4) are 6-deoxyhexoses that differ in the hydroxyl groups at C-3 and C-4.

In pLN2, oleU was replaced with the 4-ketoreductase urdZ3 creating pLNZ3. UrdZ3 participates in the biosynthesis of L-rhodinose, one of the sugars of the sugars forming part of urdamycin A. A second gene encoding a 3,4-dehydratase, urdQ from the same cluster, was inserted into pLNZ3 giving rise to the derivative pLNRHO.

The construct pLNRHO gave rise to a new HPLC peak in both reporter systems S. albus 16F4 and S. albus GB16 (described in Example 1). This new compound was consistent with L-rhodinosyl-tetracenomycin C (LRHO-TCMC) in terms of HPLC mobility and absorption spectrum when compared with the pure compound used as standard (Table 3). MALDI-TOF analysis of this compound, in the negative mode, showed a molecular peak at m/z 571.0760, consistent with LRHO-TCMC.

The construct pLNRHO gave rise to a new HPLC peak in the reporter system S. lividans NAB5 fed with EB. This new compound was consistent with a peak corresponding to L-rhodinosyl-EB (LRHO-EB) in terms of HPLC mobility, absorption spectrum and LCMS analysis when compared with the pure compounds used as standard.

### REFERENCES

Aguirrezabalaga I., Olano, C., Allende, N., Rodriguez, L., Braña, A. F., Méndez, C. and Salas, J. A. (2000) Identification and Expression of Genes involved in Biosynthesis of L-Oleandrose and Its Intermendiate L-Olivose in the Oleandomycin Producer Streptomyces antibioticus. Antimicrob. Agents Chemother. 44:1266-1275.
Altschul S. F., Gish, W., Miller, W., Myers, E. W. and Lipman, D. J. (1990) Basic Local Alignment Search Tool. J Mol. Biol. 215: 403-410.
Bartel, P. L., Zhu, C. B., Lampel, J. S., Dosch, D. C., Connors, N. C., Strohl, W. R., Beale, J. M. and Floss, H. G. (1990) Biosynthesis of anthraquinones by interspecies cloning of actinorhodin biosynthesis genes in Streptomycetes—clarification of actinorhodin gene functions. J Bacteriol. 172:4816-4826.
Bate, N., Butler, A. R., Smith, I. P. and Cundliffe, E. (2000) The mycarose-biosynthetic genes of Streptomyces fradiae, producer of tylosin. Microbiology UK 146: 139-146.
Bibb, M. J., Biró, S., Motamedi, H., Collins, J. F. and Hutchinson, C. R. (1989) Analysis of the nucleotide sequence of the Streptomyces glaucescens tcml genes provides key information about the enzymology of polyketide antibiotic biosynthesis. EMBO J. 8:2727-2736.
Blanco, G., Patallo, E. P., Braña, A. F., Trefzer, A., Bechthold, A., Rohr, J., Méndez, C. and Salas, J. A. (2001) Identification of a sugar flexible glycosyltransferase from Streptomyces olivaceus, the producer of the antitumor polyketide elloramycin. Chem. Biol. 8: 253-263.
Borisova, S. A., Zhao, L., Sherman, D. H. and Liu, H. (1999) Biosynthesis of desosamine: construction of a new macrolide carrying a genetically designed sugar moiety. Org. Lett. 1:133-136.
Bullock W. O., Fernandez, J. M. and Short, J. M. (1987) XL1-blue: A High Efficiency Plasmid Transforming RecA Escerichia coli Strain with β-Galactosidase Selection, Product Application Focus, Biotechniques 5: 376-379.
Chater, K. F. and Davis, N. K. (1990) Spore color in Streptomyces coelicolor A3(2) involves the developmentally regulated synthesis of a compound biosynthetically related to polyketide antibiotics. Mol. Microbiol. 4:1679-1691.
Cortés J., Haydock, S. F., Roberts, G. A., Bevitt, D. J. and Leadlay, P. F. (1990) An unusually large multifunctional polypeptide in the erythromycin producing polyketide synthase of Saccharopolyspora erythraea. Nature 348:176-178.
Cortés, J., Velasco, J., Foster, G., Blackaby, A. P., Rudd, B. A. M. and Wilkinson, B. (2002) Identification and cloning of a type III polyketide synthase required for diffusible pigment biosynthesis in Saccharopolyspora erythraea. Mol. Microbiol. 44:1213-1224.
Devereux, J., Haeberli, P. and Smithies, O. (1984) A comprehensive set of sequence analysis programs for the VAX. Nucleic Acids Res. 12: 387-395.
Dhillon, N., Hale, R. S., Cortés, J. and Leadlay, P. F. (1989) Molecular characterization of a gene from Saccharopolyspora erythraea (Streptomyces erythraeus) which is involved in erythromycin biosynthesis. Mol. Microbiol. 3:1405-1414.
Donadio, S., Staver, M. J., McAlpine, J. B., Swanson, S. J. and Katz, L. (1991) Modular organization of genes required for complex polyketide biosynthesis. Science 252:675-679.
Donadio, S., McAlpine, J. B., Sheldon, P. J., Jackson, M., and Katz, L. (1993) An erythromycin analog produced by reprogramming of polyketide synthesis Proc. Nat. Acad. Sci. USA 90:7119-7123.
Doumith, M., Legrand, R., Lang, C., Salas, J. A. and Raynal, M. C. (1999) Interspecies complementation in Saccharopolyspora erythraea: elucidation of the function of olep1, oleG1 and oleG2 from the oleandomycin biosynthetic gene cluster of Streptomyces antibioticus and generation of new erythromycin derivatives. Mol. Microbiol. 34:1039-1048.
Du, L., Sánchez, C. and Shen, B. (2001) Hybrid peptide-polyketide natural products: biosynthesis and prospects toward engineering novel molecules. Metab. Eng. 3:78-95.
Ferrer, J. L., Jez, J. M., Bowman, M. E., Dixon, R. A. and Noel, J. P. (1999) Structure of chalcone synthase and the molecular basis of plant polyketide biosynthesis. Nat. Str. Biol. 6:775-784.
Fernández, E, Weissbach, U., Sánchez-Reillo, C., Braña, A. F., Médndez, C. and Salas, J. A. (1998) Identification of Two Genes from Streptomyces argillaceus Encoding Glycosyltransferases Involved in Transfer of a Disaccharide During Biosynthesis of the Antitumor Drug Mithramycin. J Bacteriol. 180: 4929-4937. Fernández-Lozano, M.-J., Remsing, L. L., Quirós, L. M., Braña, A. F., Fernández, E., Sánchez, C., Méndez, C., Rohr, J. and Salas, J. A. (2000) Characterization of Two Polyketide Methyltransferases Involved in the Biosynthesis of the Antitumor Drug Mthramycin by Streptomyces argillaceus. J Biol. Chem. 275:3065-3074.
Fernández-Moreno, M. A., Martinez, E., Boto, L., Hopwood, D. A. and Malpartida, F. (1992) Nucleotide Sequence and Deduced Functions of a Set of Cotranscribed Genes of Streptomyces coelicolor A3(2) Including the Polyketide Synthase for the Antibiotic Actinorhodin. J Biol. Chem. 267:19278-19290.
Fouces, R., Mellado, E., Díez, B. and Barredo, J. L. (1999) The tylosin biosynthetic cluster from Streptomyces fradiae: genetic organization of the left region. Microbiology UK 145: 855-868.
Funa, N., Ohnishi, Y., Fujii, I., Shibuya, M., Ebizuka, Y. and Horinouchi, S. (1999) A New Pathway for Polyketide Biosynthesis in Microorganisms. Nature 400:897-899.
Hutchinson, C. R. and Fujii, I. (1995) Polyketide synthase gene manipulation: a structure-function approach in engineering novel antibiotics. Annu. Rev. Microbiol. 49:201-238.
Gaisser, S., Böhm, G. A., Cortés, J. and Leadlay, P. F. (1997) Analysis of seven genes from the eryAI-eryK region of the erythromycin biosynthetic gene cluster in Saccharopolyspora erythraea. Mol. Gen. Genet. 256:239-251.
Gaisser, S., Böhm, G. A., Doumith, M., Raynal, M. C., Dhillon, N., Cortés, J. and Leadlay, P. F. (1998) Analysis of eryBI eryBIII and eryBVII from the erythromycin biosynthetic gene cluster in Saccharopolyspora erythraea. Mol. Gen. Genet. 258:78-88.
Haydock, S. F., Dowson, J. A., Dhillon, N., Roberts, G. A., Cortés, J. and Leadlay, P. F. (1991) Cloning and sequence analysis of genes involved in erythromycin biosynthesis in Saccharopolyspora erythraea: sequence similarities between EryG and a family of S-adenosylmethionine-dependent methyltransferases. Mol. Gen. Genet. 230:120-128.
Jez, J. M., Austin, M. B., Ferrer, J. L., Bowman, M. E., Schroder, J. and Noel, J. P. (2000) Structural control of polyketide formation in plant-specific polyketide synthases. Chem. Biol. 7:919-930.
Kieser, T., Bibb, M. J., Buttner, M. J., Charter, K. F. and Hopwood, D. A. (2000) Practical Streptomyces Genetics, John Innes Foundation, Norwich.
Liu, H.-W. and Thorson, J. S. (1994) Pathways and mechanisms in the biogenesis of novel deoxysugars by bacteria. Annu. Rev. Microbiol. 48:223-256.
Madduri, K., Kennedy, J., Rivola, G., Inventi-Solari, A., Filippini, S., Zanuso, G., Colombo, A. L., Gewain, K. M., Occi, J. L., MacNeil, D. J. and Hutchinson, C. R. (1998) Production of the antitumor drug epirubicin (4′-epidoxorubicin) and its precursor by a genetically engineered strain of Streptomyces peucetius. Nature Biotechnology 16:69-74.
Meurer, G., Gerlitz, M., Wendt-Pienkowski, E., Vining, L. C., Rohr, J. and Hutchinson, C. R. (1997) Iterative type II polyketide synthases, cyclases and ketoreductases exhibit context-dependent behavior in the biosynthesis of linear and angular decapolyketides. Chem. Biol. 4:433-443.
Olano, C., Rodriguez, A. M., Michel, J. M., Méndez, C., Raynal, M. C. and Salas, J. A. (1998) Analysis of a Streptomyces antibioticus chromosomal region involved in oleandomycin biosynthesis, which encodes two glycosyltransferases responsible for glycosylation of the macrolactone ring. Mol. Gen. Genet. 259: 299-308.
Otten, S. L., Gallo, M. A., Madduri, K., Liu, X. C. and Hutchinson, C. R. (1997) Cloning and characterization of the Streptomyces peucetius dnmZUV genes encoding three enzymes required for biosynthesis of the daunorubicin precursor thymidine diphospho-L-daunosamine. J Bacteriol. 179:4446-4450.
Quirós L. M., Aguirrezabalaga, I., Olano, C., Méndez, C. and Salas, J. A. (1998) Two Glycosyltransferases and a Glycosidase are Involved in Oleandomycin Modification During Its Biosynthesis by Streptomyces antibioticus. Mol. Microbiol. 28:1177-1185.
Rawlings, B. J. (2001a) Type I polyketide biosynthesis in bacteria (Part A—erythromycin biosynthesis). Nat. Prod. Rep. 18:190-227.
Rawlings, B. J. (2001b) Type I polyketide biosynthesis (Part B). Nat. Prod. Rep. 18:231-281.
Rawlings, B. J. (1999) Biosynthesis of polyketides (other than actinomycete macrolides). Nat. Prod. Rep. 16:425-484.
Rodriguez, L., Oelkers, C., Aguirrezabalaga, I., Braña, A. F., Rohr, J., Mendez, C. and Salas, J. A. (2000) Generation of Hybrid Elloramycin Analogs by Combinatorial Biosynthesis Using Genes From Anthracycline-Type and Macrolide Biosynthetic Pathways. J. Mol. Microbiol. Biotechnol., 2: 271-6
Rodriguez, L., Rodriguez, D., Olano, C., Braña, A. F., Méndez, C. and Salas, J. A. (2001) Functional analysis of OleY L-oleandrosyl 3-O-methyltransferase of the oleandomycin biosynthetic pathway in Streptomyces antibioticus. J Bacteriol. 183:5358-5363.
Rodriguez, L., Aguirrezabalaga, I., Allende, N., Braña, A. F., Méndez, C. and Salas, J. A. (2002) Engineering deoxysugar biosynthetic pathways from antibiotic-producing microorganisms: a tool to produce novel glycosylated bioactive compounds. Chem. Biol. 9:721-729.
Rix, U., Fischer, C., Remsing, L. L. and Rohr, J. (2002) Modification of post-PKS tailoring steps through combinatorial biosynthesis. Nat Prod Rep. 19:542-80.
Salah-Bey, K., Doumith, M., Michel, J. M., Haydock, S., Cortés, J., Leadlay, P. F. and Raynal, M. C. (1 998) Targeted gene inactivation for the elucidation of deoxysugar biosynthesis in the erythromycin producer Saccharopolyspora erythraea. Mol. Gen. Genet. 257:542-553.
Salas, J. A. and Méndez, C. (2001). Altering the glycosylation pattern of bioactive compounds. Trends Biotech. 19:449-456.
Sambrook, J., Fritsch, E. F. and Maniatis, T. (1989) Moleculr Cloning: A Laboratory manual, 2nd Ed., Cold Spring Harbor Press, N.Y.
Sanger, F., Nicklen, S., and Coulsen, A. R. (1977) DNA Sequencing with Chain Terminating Inhibitors. Proc. Natl. Acad. Sci. USA. 74: 5463-5467.
Shen, B., Summers, R. G., Wendt-Pienkowski, E. and Hutchinson, C. R. (1995) The Streptomyces glaucescens tcmKL polyketide synthase and tcmN polyketide cyclase genes govern the size and shape of aromatic polyketides. J. Am. Chem. Soc. 117:6811-6821.
Sherman, D. H., Malpartida, F., Bibb, M. J., Kieser, H. M. and Hopwood, D. A. (1989) Structure and deduced function of the granaticin-producing polyketide synthase gene cluster of Streptomyces violaceoruber Tü22. EMBO J. 8:2717-2725.
Solenberg, P. J., Matsushima, P., Stack, D. R., Wilkie, S. C., Thompson, R. C. and Baltz, R. H. (1997) Production of hybrid glycopeptide antibiotics in vitro and in Streptomyces toyocaensis. Chem. Biol. 4:195-202.
Summers, R. G., Donadio, S., Staver, M. J., Wendt-Pienkowski, E., Hutchinson, C. R. and Katz, L. (1997) Sequencing and mutagenesis of genes from the erythromycin biosynthetic gene cluster of Saccharopolyspora erythraea that are involved in L-mycarose and D-desosamine production. Microbiology UK 143: 3251-3262.
Trefzer, A., Salas, J. A. and Bechthold, A. (1999) Genes and enzymes involved in deoxysugar biosynthesis in bacteria. Nat. Prod. Reports 16:283-299.
Vara, J., Lewandowska-Skarbek, M., Wang, Y. G., Donadio, S. and Hutchinson, C. R. (1989) Cloning of genes governing the deoxysugar portion of the erythromycin biosynthesis pathway in Saccharopolyspora erythraea (Streptomyces erythreus) J. Bacteriol. 171: 5872-5881.
Von Dören, H., Kéller, U. and Zocher, R. (1997) Multifunctional peptide synthetases. Chem. Rev. 97:2675-2705.
Wohlert, S. E., Lomovskaya, N., Kulowski, K., Fonstein, L., Occi, J. L., Gewain, K. M., MacNeil, D. J. and Hutchinson, C. R. (2001) Insights about the biosynthesis of the avermectin deoxysugar L-oleandrose through heterologous expression of Streptomyces avermitilis deoxysugar genes in Streptomyces lividans. Chem. Biol. 8:681-700.

Zhao, L. S., Ahlert, J., Xue, Y. Q., Thorson, J. S., Sherman, D. H. and Liu, H. W. (1999) Engineering a methymycin/pikromycin-calicheamicin hybrid: construction of two new macrolides carrying a designed sugar moiety. J. Am. Chem. Soc. 121:9881-9882.

TABLE 1
Genes used for the construction of cassette plasmids

| Gene | Microorganism | Antibiotic | Sugar pathway | Proposed function | Reference |
|---|---|---|---|---|---|
| oleS | S. antibioticus | Oleandomycin | L-oleandrose and | Glucose synthase | Agui., 2000 |
| | | | D-desosamine | | |
| oleE | S. antibioticus | Oleandomycin | L-oleandrose and | Glucose 4,6-dehydratase | Agui 2000 |
| | | | D-desosamine | | |
| oleL | S. antibioticus | Oleandomycin | L-oleandrose | 3,5-epimerase | Agui, 2000 |
| oleV | S. antibioticus | Oleandomycin | L-oleandrose | 2,3-dehydratase | Agui, 2000 |
| oleW | S. antibioticus | Oleandomycin | L-oleandrose | 3-ketoreductase | Agui 2000 |
| oleU | S. antibioticus | Oleandomycin | L-oleandrose | 4-ketoreductase | Agui2000 |
| oleY | S. antibioticus | Oleandomycin | L-oleandrose | 3-O-methyltransferase | Olano, 1998, |
| | | | | | Rodriguez 2001 |
| eryBIV | Sacc. erythraea | Erythromycin | L-mycarose | 4-ketoreductase | Gaisser Summer |
| snogC | S. nogalater | Nogalamycin | L-nogalose | 4-ketoreductase | AAF01815 |
| dnmV | S. peucetius | Daunorubicin | L-daunosamine | 4-ketoreductase | Otten |
| tylD | S. fradiae | Tylosin | 6-deoxy-D-allose | 4-ketoreductase | Fouces; Bate |

TABLE 2

Synthetic oligonucleotides used for PCR
amplification of individual sugar biosynthetic genes

| | | | |
|---|---|---|---|
| Primers | Gene | DNA sequence (5′-3′) | Restriction sites |

| | | | |
|---|---|---|---|
| 5′-7U | oleU | AGAGAAGCTTACTAGTGCCCCCCCGAGAGGC | HindIII, SpeI |
| | | AGCGGCCCATG | |
| 3′-7L | | AGTCTAGAGCTAGCTCATGCTGCTCCTCGGC | XbaI, NheI |
| | | GGGTCGGTGGG | |
| 5′-14U | oleY | AGAGAAGCTTGCTAGCCGGACCACGCGAAGG | HindIII, NheI |
| | | ACCTTTCACATG | |
| 3′-14L | | AGTCTAGATTAATTAAGCTAGTTGTCGTTCC | XbaI, Pad |
| | | AGAACGGCTCCCGG | |
| 5′-1α | oleV | ACGTAAGCTTCCTAGGGCGCCGTCCTGGATC | HindIII, AvrII |
| | | ACAATG | |
| 3′-1β | | ACGTCTAGAGTTAACCTTCGGGGTGCTCAGC | XbaI, HpaI |
| | | TCAGG | |
| 5′-15α | oleW | ACGTAAGCTTGTTAACCCGAAGGGAACCCCA | HindIII, HpaI |
| | | TGCCC | |
| 3′-15β | | ACGTCTAGAACTAGTGCATCAGCACCAGCGC | XbaI, SpeI |
| | | ACCCG | |
| 5′-VU | dnmV | AGAGAAGCTTACTAGTGGGTCACGCGGAGAC | HindIII, SpeI |
| | | GGGTGAGGCGGACATG | |
| 3′-VL | | AGTCTAGAGCTAGCCTAGGCCGGGGCGCCGT | XbaI, NheI |
| | | GCGCGGTCC | |
| 5′-SU | snogC | AGAGAAGCTTACTAGTGCGTGCTGGTCACCG | HindIII, SpeI |
| | | GAGGAGAAGGCATG | |
| 3′-SL | | AGTCTAGAGCTAGCTCACGTCCCCGTCAGGT | XbaI, NheI |
| | | TCCTGAGGCGC | |
| 5′-TU | tylD | AGAGAAGCTTACTAGTCGCTCCGCCCACCGG | HindIII, SpeI |
| | | GCGCGACGCATG | |
| 3′-TL | | AGTCTAGAGCTAGCTCACGAGTGGGGTGCCC | XbaI, NheI |
| | | GCGACGGCCGG | |
| 5′-B4U | eryBIV | AGAGAAGCTTACTAGTCAGTGACGGGTTGCC | HindIII, SpeI |
| | | GCACATCGCGCTGAATG | |
| 3′-B4L | | AGTCTAGAGCTAGCCTAGTGCTCCTCGGTGG | XbaI, NheI |
| | | GGGTCAGGGCGGCC | |

TABLE 3
Plasmid constructs directing the biosynthesis of different sugars and
GTF system used to show the presence of the sugar

| | oleU | | GTF used | | |
|---|---|---|---|---|---|
| Plasmid | replaced by | Sugar expected | oleG2 | eryBV | elmGT |
| pLN2 | — | L-olivose | L-oleandrosyl-EB | L-oleandrosyl-EB | L-olivosyl-TcmC |
| | | (L-oleandrose) | | | |
| pLN2EryBIV | eryBIV | L-olivose | L-oleandrosyl-EB | L-oleandrosyl-EB* | L-digitoxyl-TcmC |
| | | (L-oleandrose) | | L-digitoxyl-EB | |
| | | | | 3-O-methyl-L- | |
| | | | | digitoxyl-EB | |
| pLN2SnogC | snogC | L-olivose | — | — | — |
| | | (L-oleandrose) | | | |
| pLN2DnmV | dnmV | L-oliose | — | — | — |
| pLN2TylD | tylD | Unknown | — | — | — |
| pLN2ΔVW | — | L-rhamnose | L-rhamnosyl-EB | — | L-rhamnosyl-TcmC |
| pLNRHO | urdZ3; | L-rhodinose | | L-rhodinose-EB | L-rhodinose-TcmC |
| | urdQ | | | | |
| pLNR | urdR | D-olivose | | D-olivosyl-EB | D-olivosyl-TcmC |
| | | (D-oleandrose) | | D-oleandrosyl-EB | |

1. A process for producing a hybrid glycosylated product by transferring one or more sugar moieties to an aglycone template, the process comprising:
(a) constructing a plasmid-based gene cassette which contains nucleic acid encoding sugar synthesis genes sufficient to direct the synthesis of a specific activated sugar, wherein at least some of said sugar synthesis genes are flanked by restriction sites,
(b) transformation of microorganism host cells: (i) with said plasmid-based gene cassette; and (ii) with nucleic acid encoding a glycosyltransferase (GT); and,
(c) providing an aglycone template to said transformed microorganism, or allowing its endogenous generation, so that the GT transfers one or more sugar moieties to the aglycone template in order to produce a hybrid glycosylated product;
wherein one or more of the sugar moieties, the aglycone template, the glycosyltransferase, the sugar synthesis genes or the host cells are heterologous to one or more of the other components.

2. A process according to claim 1, wherein each of said sugar synthesis genes in the cassette is flanked by restriction sites.

3. A process according to claim 1, wherein said restriction sites are unique restriction sites.

4. A process according to claim 1, wherein said genes, with their restriction sites if present, are generated by means of the polymerase chain reaction (PCR).

5. A process according to claim 4, wherein a multiplicity of sugar synthesis genes are generated by the PCR and combinatorially assembled to produce a multiplicity of different cassettes which are used in the transformation of respective different host cells.

6. A process according to claim 1 wherein at least some of said sugar synthesis genes have their own ribosome binding sites.

7. A process according to claim 6, wherein all of said sugar synthesis genes have their own ribosome binding sites.

8. A process according to claim 1, wherein the aglycone template and the GT are mutually heterologous.

9. A process according to claim 1, wherein the aglycone template is exogenously supplied.

10. A process according to claim 1 wherein the aglycone template comprises a polyketide.

11. A process according to claim 1 wherein the aglycone template comprises a peptide.

12. A process according to claim 1 wherein said plasmid-based gene cassette comprises some or all of the genes required for the synthesis of one or more of oleandrose, desosamine, mycarose, mycaminose, rhodinose, oliose and olivose.

13. A transformant host cell produced by transforming a precursor host cell with
(a) a plasmid-based gene expression cassette which contains nucleic acid encoding sugar synthesis genes sufficient to direct the synthesis of a specific activated sugar in those host cells, optionally operably linked under the same promoter, wherein at least some of said sugar synthesis genes in the cassette are flanked by restriction sites; and
(b) with a glycosyltransferase (GT), wherein the GT is heterologous to the host cells and is capable of transferring one or more sugar moieties to an aglycone template within the cells to produce a hybrid glycosylated product.

14. A transformant host cell according to claim 13 wherein said plasmid-based gene cassette comprises some or all of the genes required for the synthesis of one or more of oleandrose, desosamine, mycarose, mycaminose, rhodinose, oliose and olivose.

15. A process for producing a hybrid glycosylated product, the process comprising culturing the host cells according to claim 13 and isolating the product thus produced.

16. A process according to claim 15, wherein said process additionally comprises the step of supplying the aglycone template to the cells.

17. A hybrid glycosylation product as obtainable by the process of claim 1.

18. A method for screening cloned microbial glycosyltransferases for their ability to generate specific glycosylated derivatives, when supplied with an aglycone template, which method comprises placing said cloned microbial glycosyltransferases in contact with the host cells of claim 13 and observing the generation, if any, of said specific glycosylated derivatives.

19. A hybrid glycosylation product as obtainable by the process of claim 15.

20. A hybrid glycosylation product as obtainable by the process of claim 16.

21. A method for screening cloned microbial glycosyltransferases for their ability to generate specific glycosylated derivatives, when supplied with an aglycone template, which method comprises placing said cloned microbial glycosyltransferases in contact with the host cells of claim 14 and observing the generation, if any, of said specific glycosylated derivatives.
